(19) United States
(12) Patent Application Publication
(10) Pub. No.: US 2009/0153796 A1
(43) Pub. Date: Jun. 18, 2009

(54) Multi-functional optometric-ophthalmic system for testing diagnosing, or treating, vision or eyes of a subject, and methodologies thereof

(76) Inventor: Arthur Rabner, Natania (IL)

Correspondence Address: MARTIN D. MOYNIHAN d/b/a PRTSI, INC., P.O. BOX 16446, ARLINGTON, VA, 22215, US

(21) Appl. No.: 11/991,242

(22) PCT Filed: Sep. 3, 2006
(86) PCT No.: PCT/IL2006/001022

§ 371 Date: Feb. 29, 2008

Related U.S. Application Data
(60) Provisional application No. 60/596,140, filed on Sep. 2, 2005.

(51) Int. Cl. A61B 3/18; A61B 3/10; A61B 3/08; A61B 3/107; A61B 3/16; A61B 3/14; A61B 3/032

(52) U.S. Cl. 351/201; 351/211; 351/212; 600/398; 351/206; 351/203; 351/246

(57) ABSTRACT

Multi-functional optometric-ophthalmic system for testing, diagnosing, or treating, vision or eyes of a subject, and methodologies thereof. Includes: a head mountable unit, including a head mounting assembly, and at least one near eye module assembly (NEMa), mounted upon the head mounting assembly, for generating optical processes or effects which act or take place upon, and are affected by, at least one eye of the subject, and for receiving results of the optical processes or effects from the at least one eye, as part of the testing, diagnosing, or treating of the vision or eyes of the subject; and a central controlling and processing unit. Near eye module assembly includes: a micro-display (μdisplay), a first lens assembly (L1a), and a refraction correction assembly (RCa). Generally applicable for performing a wide variety of different optometric and ophthalmic tests, diagnoses, and treatments, of a subject's vision or eyes.

### FIELD AND BACKGROUND OF THE INVENTION

The present invention relates to the fields of optometry and ophthalmology, involving, associated with, or relating to, testing, diagnosing, or treating, vision or eyes of a subject, and more particularly, to a multi-functional optometric-ophthalmic system for testing, diagnosing, or treating, vision or eyes of a subject, and methodologies thereof. The present invention is generally applicable for performing a wide variety of different optometric and ophthalmic tests, diagnoses, and treatments, of a subject's vision or eyes.

Theories, principles, and practices thereof, and, related and associated applications and subjects thereof, relating to testing, diagnosing, or treating, vision or eyes of a subject, are well known and taught about in the prior art, and currently practiced in the fields of optometry and ophthalmology. For the purpose of establishing the scope, meaning, and field(s) or area(s) of application, of the present invention, the following background includes selected definitions and exemplary usages of terminology which are relevant to, and used for, disclosing the present invention.

### Optometric and Ophthalmic

Herein, in the context of the field and art of the present invention, the term ‘optometric’ generally refers to an activity, piece of equipment (system, device, apparatus, instrument), or object, used for, involving, associated with, or relating to, testing (examining), diagnosing, or treating, vision, eyes, or related structures, of a subject, for the purpose or objective of determining (i.e., diagnosing) or treating (i.e., correcting) a vision problem using lenses (i.e., in the form of glasses or contact lenses) or/and other optical aids. The term ‘ophthalmic’ generally refers to an activity, piece of equipment (system, device, apparatus, instrument), or object, used for, involving, associated with, or relating to, testing (examining), diagnosing, or treating, vision, eyes, or related structures, of a subject, for the purpose or objective of determining (i.e., diagnosing) or treating (i.e., correcting, typically by a surgical procedure) a defect, illness, or disease, of eyes or related structures.

In general, the terms optometric and ophthalmic may overlap and refer to a same or similar activity, piece of equipment, or object, however, by convention, a distinction or separation exists between these terms, whereby the term ‘ophthalmic’ is more restricted and specialized by involving, being associated with, or relating to, an activity, piece of equipment, or object, used as part of a surgical procedure for surgically correcting a defect, illness, or disease, of an eye or related structure. For the purpose of maintaining generality, while at the same time maintaining clarity of presentation and understanding of the subject matter of the present disclosure, the hyphenated (dual term) phrase ‘optometric-ophthalmic’ is generally used when referring to either an optometric activity, piece of equipment, or object, or an ophthalmic activity, piece of equipment, or object.

In spite of extensive teachings in the fields of optometry and ophthalmology, and in view of various significant limitations associated with such teachings, there is an on-going need for developing and practicing improved or/and new equipment and methodologies using thereof, for testing, diagnosing, or treating, vision or eyes of a subject.

There is thus a need for, and it would be highly advantageous to have a multi-functional optometric-ophthalmic system for testing, diagnosing, or treating, vision or eyes of a subject, and methodologies thereof.

### SUMMARY OF THE INVENTION

The present invention relates to a multi-functional optometric-ophthalmic system for testing, diagnosing, or treating, vision or eyes of a subject, and methodologies thereof. The present invention is generally applicable for performing a wide variety of different optometric and ophthalmic tests, diagnoses, and treatments, of a subject's vision or eyes.

Thus, according to the present invention, there is provided a multi-functional optometric-ophthalmic system for testing, diagnosing, or treating, vision or eyes of a subject, comprising: (a) a head mountable unit, mounted upon head of the subject, wherein the head mountable unit includes: (i) a head mounting assembly, for mounting assemblies of the system upon the head of the subject; and (ii) at least one near eye module assembly (NEMa), mounted upon the head mounting assembly, for generating optical processes or effects which act or take place upon, and are affected by, at least one eye of the subject, and for receiving results of the optical processes or effects from the at least one eye, as part of the testing, diagnosing, or treating of the vision or eyes of the subject, wherein the near eye module assembly includes: (1) a micro-display (μdisplay), for generating, and emitting, light rays which are transmitted along an incident optical path, and directed into an eye of the subject, for interacting with, and being partly reflected by, retina or/and other components of the eye; (2) a first lens assembly (L1a), for refracting the light rays generated and emitted by the micro-display into groups of parallel light rays, which are transmitted to the eye, and for refracting light rays which are reflected by the retina or/and other components of the eye; and (3) a refraction correction assembly (RCa), for correcting a wave front of the light rays paralleled by the first lens assembly, for adjusting a state of refraction of the eye, and for refracting the paralleled light rays, for regulating a state of distance perception of the eye; and (b) a central controlling and processing unit, operatively connected to the head mountable unit, for controlling and processing of functions, activities, and operations, of components of the head mountable unit.

Accordingly, by way of the near eye module assembly being a sub-combination of the head mountable unit included in the multi-functional optometric-ophthalmic system for testing, diagnosing, or treating, vision or eye of a subject, therefore, the present invention also features an optometric-ophthalmic device, corresponding to the near eye module assembly, for testing, diagnosing, or treating, vision or eye of a subject.

Thus, according to another aspect of the present invention, there is provided an optometric-ophthalmic device, for testing, diagnosing, or treating, vision or eye of a subject, comprising: a micro-display (μdisplay), for generating, and emitting, light rays which are transmitted along an incident optical path, and directed into the eye of the subject, for interacting with, and being partly reflected by, retina or/and other components of the eye; a first lens assembly (L1a), for refracting the light rays generated and emitted by the micro-display into groups of parallel light rays, which are transmitted to the eye, and for refracting light rays which are reflected by the retina or/and other components of the eye; and a refraction correction assembly (RCa), for correcting a wave front of the light rays paralleled by the first lens assembly, for adjusting a state of refraction of the eye, and for refracting the paralleled light rays, for regulating a state of distance perception of the eye; wherein the optometric-ophthalmic device is used for generating optical processes or effects which act or take place upon, and are affected by, the eye, and for receiving results of the optical processes or effects from the eye.

According to another aspect of the present invention, there is provided a method for testing, diagnosing, or treating vision or eyes of a subject, the method comprising: (a) mounting a head mountable unit upon head of the subject, wherein the head mountable unit includes: (i) a head mounting assembly, for mounting assemblies of the system upon the head of the subject; and (ii) at least one near eye module assembly (NEMa), mounted upon the head mounting assembly, for generating optical processes or effects which act or take place upon, and are affected by, at least one eye of the subject, and for receiving results of the optical processes or effects from the at least one eye, as part of the testing, diagnosing, or treating of the vision or eyes of the subject, wherein the near eye module assembly includes: (1) a micro-display (μdisplay), for generating, and emitting, light rays which are transmitted along an incident optical path, and directed into an eye of the subject, for interacting with, and being partly reflected by, retina or/and other components of the eye; (2) a first lens assembly (L1a), for refracting the light rays generated and emitted by the micro-display into groups of parallel light rays, which are transmitted to the eye, and for refracting light rays which are reflected by the retina or/and other components of the eye; and (3) a refraction correction assembly (RCa), for correcting a wave front of the light rays paralleled by the first lens assembly, for adjusting a state of refraction of the eye, and for refracting the paralleled light rays, for regulating a state of distance perception of the eye; and (b) controlling and processing of functions, activities, and operations, of components of the head mountable unit, by a central controlling and processing unit, operatively connected to the head mountable unit.

According to further characteristics in preferred embodiments of the invention described below, in the near eye module assembly, the micro-display generates, and emits, normal intensity patterns, pictures, or/and videos, which are transmitted to the eye.

According to further characteristics in preferred embodiments of the invention described below, in the near eye module assembly, the micro-display generates, and emits, short interval pulses of high intensity pattern or illumination, which are transmitted to the eye.

According to further characteristics in preferred embodiments of the invention described below, in the near eye module assembly, the short interval pulses are on order of milliseconds time duration.

According to further characteristics in preferred embodiments of the invention described below, in the near eye module assembly, the micro-display generates and emits white light rays having a spectrum including wavelengths in a range of between about 200 nanometers and about 10,000 nanometers.

According to further characteristics in preferred embodiments of the invention described below, in the near eye module assembly, the micro-display is designed, constructed, and operates, according to organic light emitting diode technology.

According to further characteristics in preferred embodiments of the invention described below, in the near eye module assembly, the micro-display has an active display area with a resolution of 900 pixels×600 pixels, wherein pixel size is 15 microns×15 microns.

According to further characteristics in preferred embodiments of the invention described below, in the near eye module assembly, each pixel is partitioned into three sub-pixels, each of size 5 microns×15 microns, for converting white light rays to colored light rays, and for testing vision acuities higher than 6/6 vision acuity based on a design requirement of the 6/6 vision acuity.

According to further characteristics in preferred embodiments of the invention described below, in the near eye module assembly, the first lens assembly includes an in/out moving and positioning sub-assembly for moving and positioning of the first lens assembly in or out of the incident optical path directed into the eye.

According to further characteristics in preferred embodiments of the invention described below, in the near eye module assembly, the refraction correction assembly includes components and functionalities thereof, according to a spherical type correction, a cylindrical type correction, a prismatic type correction, or a combination thereof.

According to further characteristics in preferred embodiments of the invention described below, in the near eye module assembly, according to a the spherical type correction, there is changing optical distance extending between the micro-display and the first lens assembly, along the incident optical path directed into the eye.

According to further characteristics in preferred embodiments of the invention described below, in the near eye module assembly, another function of the refraction correction assembly is for regulating monocular distance perception of virtual objects perceived by the subject.

According to further characteristics in preferred embodiments of the invention described below, the near eye module assembly includes a red-green-blue filter assembly (RGBFa) for converting white light rays generated by, and emitted from, the micro-display, to colored light rays which travel along the incident optical path directed into the eye, wherein the red-green-blue filter assembly covers about 10% of total active display area of the micro-display.

According to further characteristics in preferred embodiments of the invention described below, the near eye module assembly includes a micro-display filters assembly (μDFa) for selectively filtering the light rays generated and emitted by the micro-display.

According to further characteristics in preferred embodiments of the invention described below, the near eye module assembly includes a second lens assembly (L2a) for increasing optical power over that provided by the first lens assembly, wherein the second lens assembly includes an in/out moving and positioning sub-assembly, for moving and positioning of the second lens assembly in or out of the incident optical path directed into the eye.

According to further characteristics in preferred embodiments of the invention described below, the near eye module assembly includes a mirror for changing direction of the light rays generated and emitted by the micro-display, and for serving as a controllable gate or barrier, for controllably gating or blocking the eye from being exposed to a local environment external to, and outside of, the near eye module assembly.

According to further characteristics in preferred embodiments of the invention described below, the near eye module assembly includes a mirror position regulator (MPR) for regulating or changing position of the mirror spanning between a fully open mirror position and a fully closed mirror position.

According to further characteristics in preferred embodiments of the invention described below, the near eye module assembly includes a beam splitter for splitting the light rays generated and emitted by the micro-display into two groups of light rays.

According to further characteristics in preferred embodiments of the invention described below, the near eye module assembly includes a pinhole shutter and airpuff/ultrasound assembly for controlling intensity of a portion of the light rays generated and emitted by the micro-display, and, for applying an air pressure wave or an ultrasound pressure wave onto cornea of the eye.

According to further characteristics in preferred embodiments of the invention described below, in the near eye module assembly, the pinhole shutter and airpuff/ultrasound assembly includes an ultrasound wave transducer, for generating and distributing the ultrasound pressure wave to the cornea, and for sensing a response by the cornea to the ultrasound pressure wave.

According to further characteristics in preferred embodiments of the invention described below, the near eye module assembly includes a frontal distance regulator (FDR) for regulating or changing optical distance extending between the pinhole shutter and airpuff/ultrasound assembly and the eye, along the incident optical path directed into the eye.

According to further characteristics in preferred embodiments of the invention described below, the near eye module assembly includes a third lens assembly (L3a) for increasing optical power over that provided by the first lens assembly, wherein the third lens assembly includes an in/out moving and positioning sub-assembly, for moving and positioning of the third lens assembly in or out of a reflection optical path directed out of the eye.

According to further characteristics in preferred embodiments of the invention described below, the near eye module assembly includes an imager filters assembly for selectively filtering light rays reflected by the retina or/and other components of the eye.

According to further characteristics in preferred embodiments of the invention described below, the near eye module assembly includes an imager for capturing still or video patterns or images reflected by the retina or/and other components of the eye.

According to further characteristics in preferred embodiments of the invention described below, the near eye module assembly includes an image distance regulator (IDR) for regulating or changing optical distance extending between the first lens assembly and the imager, along a reflection optical path directed out of the eye.

According to further characteristics in preferred embodiments of the invention described below, the near eye module assembly includes a micro-display distance regulator (μDDR) for regulating or changing optical distance extending between the micro-display and the first lens assembly, along the incident optical path directed into the eye.

According to further characteristics in preferred embodiments of the invention described below, in the near eye module assembly, the regulating or changing of the optical distance is performed for: (1) matching optical power provided by the first lens assembly along the incident optical path, or (2) compensating a myopic or hyperopic refractive condition of the eye, or (3) emulating distance of perception by the subject of a virtual object displayed by the micro-display, or (4) adjusting and attaining a fine focal distance of the light rays passing through a filter assembly, or a combination thereof.

According to further characteristics in preferred embodiments of the invention described below, the near eye module assembly includes a reality window is for exposing the eye to a real environment external to, and outside of, the near eye module assembly.

According to further characteristics in preferred embodiments of the invention described below, the near eye module assembly includes a micro-display calibration sensor assembly (μDCSa) for measuring, and testing, emission power of the micro-display, and for deactivating the micro-display.

According to further characteristics in preferred embodiments of the invention described below, the near eye module assembly includes a mobile imaging assembly for imaging anterior parts of the eye, and for imaging facial anatomical features and characteristics in an immediate region of the eye.

According to further characteristics in preferred embodiments of the invention described below, in the near eye module assembly, the mobile imaging assembly includes: (1) a multi-spectral illumination source, (2) an imager, and (3) an electronically adjustable focus lens.

According to further characteristics in preferred embodiments of the invention described below, the head mountable unit includes at least one multi-axis moving and positioning assembly, for moving and positioning of the near eye module assembly relative to the eye for up to six degrees of freedom, including linear translation along x-axis, y-axis, or/and z-axis, or/and rotation around the x-axis, the y-axis, or/and the z-axis.

According to further characteristics in preferred embodiments of the invention described below, the head mountable unit includes at least one secondary fixation pattern assembly, for generating a fixation pattern for the eye, wherein the secondary fixation pattern assembly includes: (1) an emission pattern sub-assembly, (2) a secondary fixation pattern refraction correction sub-assembly, and (3) a refractive surface mirror.

According to further characteristics in preferred embodiments of the invention described below, the head mountable unit includes at least one multi-axis moving and positioning assembly, for moving and positioning of the secondary fixation pattern assembly relative to the eye for up to six degrees of freedom, including linear translation along x-axis, y-axis, or/and z-axis, or/and rotation around the x-axis, the y-axis, or/and the z-axis.

According to further characteristics in preferred embodiments of the invention described below, the head mountable unit includes at least one fixed imaging assembly, for observing and imaging in and around immediate regions of the eye.

According to further characteristics in preferred embodiments of the invention described below, the head mountable unit includes a sensoric electrodes assembly, for sensing a visual evoked potential in visual cortex area of brain of the subject.

The present invention is implemented by performing steps, sub-steps, and procedures, in a manner selected from the group consisting of manually, semi-automatically, fully automatically, and a combination thereof, involving use and operation of system units, system sub-units, devices, assemblies, sub-assemblies, mechanisms, structures, components, and elements, and, peripheral equipment, utilities, accessories, and materials, in a manner selected from the group consisting of manually, semi-automatically, fully automatically, and a combination thereof. Moreover, according to actual steps, sub-steps, procedures, system units, system sub-units, devices, assemblies, sub-assemblies, mechanisms, structures, components, and elements, and, peripheral equipment, utilities, accessories, and materials, used for implementing a particular embodiment of the disclosed invention, the steps, sub-steps, and procedures, are performed by using hardware, software, or/and an integrated combination thereof, and the system units, sub-units, devices, assemblies, sub-assemblies, mechanisms, structures, components, and elements, and, peripheral equipment, utilities, accessories, and materials, operate by using hardware, software, or/and an integrated combination thereof.

In particular, software used for implementing the present invention includes operatively connected and functioning written or printed data, in the form of software programs, software routines, software sub-routines, software symbolic languages, software code, software instructions or protocols, software algorithms, or/and a combination thereof. In particular, hardware used for implementing the present invention includes operatively connected and functioning electrical, electronic or/and electromechanical system units, sub-units, devices, assemblies, sub-assemblies, mechanisms, structures, components, and elements, and, peripheral equipment, utilities, accessories, and materials, which may include one or more computer chips, integrated circuits, electronic circuits, electronic sub-circuits, hard-wired electrical circuits, or/and combinations thereof, involving digital or/and analog operations. Accordingly, the present invention is implemented by using an integrated combination of the just described software and hardware.

### BRIEF DESCRIPTION OF THE DRAWINGS

The present invention is herein described, by way of example only, with reference to the accompanying drawings. With specific reference now to the drawings in detail, it is stressed that the particulars shown are by way of example and for purposes of illustrative description of the preferred embodiments of the present invention only, and are presented in the cause of providing what is believed to be the most useful and readily understood description of the principles and conceptual aspects of the present invention. In this regard, no attempt is made to show structural details of the present invention in more detail than is necessary for a fundamental understanding of the invention, the description taken with the drawings making apparent to those skilled in the art how the several forms of the invention may be embodied in practice. In the drawings:

FIG. 1 is a block diagram illustrating an exemplary preferred embodiment of the system, multi-functional optometric-ophthalmic system 10, for testing, diagnosing, or treating, vision or eyes of a subject 12, by an operator 15, wherein the system includes main components of: a head mountable unit 14, and a central controlling and processing unit 16, and wherein the head mountable unit 14 includes main components of: a head mounting assembly 18, and at least one near eye module assembly (NEMa), e.g., near eye module assembly (NEMa) 20a and near eye module assembly (NEMa) 20b, in accordance with the present invention;

FIG. 2 is a schematic diagram illustrating an exemplary preferred embodiment of implementing multi-functional optometric-ophthalmic system 10, for testing, diagnosing, or treating, vision or eyes of subject 12, by operator 15, in accordance with the present invention;

FIGS. 3a, 3b, and 3c, are schematic diagrams illustrating close-up (partly exposed) side view (FIG. 3a), front view (FIG. 3b), and top view (FIG. 3c), of an exemplary specific preferred embodiment of near eye module assembly (NEMa) 20 (i.e., near eye module assembly (NEMa) 20a or near eye module assembly (NEMa) 20b, of FIG. 1), and components thereof, as part of multi-functional optometric-ophthalmic system 10 illustrated in FIGS. 1 and 2, in accordance with the present invention;

FIGS. 4a, 4b, and 4c, are schematic diagrams illustrating front and side views of different exemplary specific preferred embodiments of pinhole shutter and airpuff/ultrasound assembly 220, and components thereof, as part of near eye module assembly (NEMa) (20 in FIGS. 3a, 3b, and 3c; 20a or 20b, in FIG. 1), of multi-functional optometric-ophthalmic system 10 illustrated in FIGS. 1 and 2, in accordance with the present invention;

FIG. 5a is a schematic diagram illustrating an optical diagram showing an exemplary calculation of size dimension, h, of fine detail projected onto a fovea of an eye, corresponding to 1′ angle of view, regarding the 6/6 vision acuity (VA) design requirement of the near eye module assembly (NEMa) (20 in FIGS. 3a, 3b, and 3c; 20a or 20b, in FIG. 1), of the multi-functional optometric-ophthalmic system (10 illustrated in FIGS. 1 and 2), in accordance with the present invention;

FIG. 5b, a schematic diagram illustrating an optical diagram showing an exemplary calculation of focal distance, fₗₑₙₛ, of first lens assembly (L1a) 216 used with micro-display (μdisplay) 202, as another design requirement of the near eye module assembly (NEMa) (20 in FIGS. 3a, 3b, and 3c; 20a or 20b, in FIG. 1), of the multi-functional optometric-ophthalmic system (10 illustrated in FIGS. 1 and 2), in accordance with the present invention;

FIG. 5c is a schematic diagram illustrating different exemplary specific embodiments or configurations of optotypes (generated by micro-display (μdisplay) 202), used for testing vision acuities higher than 6/6, based on the 6/6 vision acuity design requirement illustrated in FIGS. 5a and 5b, in accordance with the present invention;

FIG. 6a is a schematic diagram illustrating a calculation of the field of view (FOV), based on the 6/6 vision acuity design requirement illustrated in FIGS. 5a and 5b, in accordance with the present invention;

FIG. 6b is a schematic diagram illustrating an exemplary calculation of field of a view (FOV), without the 6/6 vision acuity design requirement shown in FIGS. 5a and 5b, in accordance with the present invention;

FIG. 6c is a schematic diagram illustrating an exemplary specific embodiment of an optical configuration suitable for corneal imaging, using near eye module assembly (NEMa) (20 in FIGS. 3a, 3b, and 3c; 20a or 20b, in FIG. 1), of the multi-functional optometric-ophthalmic system (10 illustrated in FIGS. 1 and 2), in accordance with the present invention;

FIG. 7 is a schematic diagram illustrating a side view of an exemplary specific preferred embodiment of secondary fixation pattern assembly (SFPa) 24, and components thereof, as part of head mountable unit 14, of multi-functional optometric-ophthalmic system 10 illustrated in FIGS. 1 and 2; in accordance with the present invention;

FIG. 8 is a schematic diagram illustrating a top view of an exemplary specific preferred embodiment particularly showing relative positions, and fields of view 330 and 332, of mobile imaging assembly 246 and fixed imaging assembly 28, in relation to facial anatomical features and characteristics in the immediate region of eye 102a of subject 12, for imaging thereof via multi-functional optometric-ophthalmic system 10 illustrated in FIGS. 1 and 2; in accordance with the present invention;

FIGS. 9a and 9b are schematic diagrams illustrating definition of the geometrical center of the eye 602, the eye opening contour 606, and the inter-pupillary normal distance (IPND) 608, in accordance with the present invention;

FIG. 10a is a schematic diagram illustrating an example of a configuration of positions of the near eye module assembly (NEMa) 20 in combination with the secondary fixation pattern assembly (SFPa) 24, for implementation of the ‘Retinal Photography and Scanning for Ultra-Wide Field of View’ procedure, in accordance with the present invention;

FIG. 10b is a schematic diagram illustrating θ 650 and Φ 652 retina image scans that creates a combined field of view (CFOV) 654 solid angle, as used in the ‘Retinal Photography and Scanning for Ultra-Wide Field of View’ procedure, in accordance with the present invention;

FIGS. 11a, 11b, and 11c are schematic diagrams illustrating positions of the near eye module assemblies (NEMa) 20a and 20b for emulation in a binocular mode of perceiving virtual objects at different distances and locations from the subject 12, in accordance with the present invention;

FIGS. 12a, 12b, 12c, and 12d are schematic diagrams illustrating inability of convergence or divergence of the left eye 102a of the subject 12, together with emulation of a base in a prism 608 (FIG. 12b) and a base out of a prism 614 (FIG. 12d), using shift of the near eye module assembly (NEMa) 20a, for the subject 12 performing a binocular fixation, in accordance with the present invention;

FIGS. 13a, 13b, 13c, 13d, and 13e are schematic diagrams illustrating a cover test procedure sequence, in accordance with the present invention;

FIGS. 14a and 14b are schematic diagrams illustrating a progressive projection of patterns onto the cornea 152, in accordance with the present invention; and

FIGS. 15a, 15b, 15c, and 15d are schematic diagrams illustrating the astigmatism test procedure sequence, using an embodiment of the refraction correction assembly (RCa) 218 absent of cylindrical correction optics, in accordance with the present invention.

### DESCRIPTION OF THE PREFERRED EMBODIMENTS

The present invention relates to a multi-functional optometric-ophthalmic system for testing, diagnosing, or treating, vision or eyes of a subject, and methodologies thereof. The present invention is generally applicable for performing a wide variety of different optometric and ophthalmic tests, diagnoses, and treatments, of a subject's vision or eyes.

The multi-functional optometric-ophthalmic system for testing, diagnosing, or treating, vision or eyes of a subject, of the present invention, includes the following main components and functionalities thereof: (a) a head mountable unit, mounted upon head of the subject, wherein the head mountable unit includes: (i) a head mounting assembly, for mounting assemblies of the system upon the head of the subject; and (ii) at least one near eye module assembly (NEMa), mounted upon the head mounting assembly, for generating optical processes or effects which act or take place upon, and are affected by, at least one eye of the subject, and for receiving results of the optical processes or effects from the at least one eye, as part of the testing, diagnosing, or treating of the vision or eyes of the subject, wherein the near eye module assembly includes: (1) a micro-display (μdisplay), for generating, and emitting, light rays which are transmitted along an incident optical path, and directed into an eye of the subject, for interacting with, and being partly reflected by, retina or/and other components of the eye; (2) a first lens assembly (L1a), for refracting the light rays generated and emitted by the micro-display into groups of parallel light rays, which are transmitted to the eye, and for refracting light rays which are reflected by the retina or/and other components of the eye; and (3) a refraction correction assembly (RCa), for correcting a wave front of the light rays paralleled by the first lens assembly, for adjusting a state of refraction of the eye, and for refracting the paralleled light rays, for regulating a state of distance perception of the eye; and (b) a central controlling and processing unit, operatively connected to the head mountable unit, for controlling and processing of functions, activities, and operations, of components of the head mountable unit.

Accordingly, by way of the near eye module assembly being a sub-combination of the head mountable unit included in the multi-functional optometric-ophthalmic system for testing, diagnosing, or treating, vision or eye of a subject, therefore, the present invention also features an optometric-ophthalmic device, corresponding to the near eye module assembly, for testing, diagnosing, or treating, vision or eye of a subject.

The optometric-ophthalmic device for testing, diagnosing, or treating, vision or eye of a subject, herein, also referred to as the near eye module assembly (NEMa) device, of the present invention, includes the main components and functionalities thereof: a micro-display (μdisplay), for generating, and emitting, light rays which are transmitted along an incident optical path, and directed into the eye of the subject, for interacting with, and being partly reflected by, retina or/and other components of the eye; a first lens assembly (L1a), for refracting the light rays generated and emitted by the micro-display into groups of parallel light rays, which are transmitted to the eye, and for refracting light rays which are reflected by the retina or/and other components of the eye; and a refraction correction assembly (RCa), for correcting a wave front of the light rays paralleled by the first lens assembly, for adjusting a state of refraction of the eye, and for refracting the paralleled light rays, for regulating a state of distance perception of the eye; wherein the optometric-ophthalmic device is used for generating optical processes or effects which act or take place upon, and are affected by, the eye, and for receiving results of the optical processes or effects from the eye.

The corresponding method for testing, diagnosing, or treating, vision or eyes of a subject, of the present invention, includes the following main steps or procedures, and, components and functionalities thereof: (a) mounting a head mountable unit upon head of the subject, wherein the head mountable unit includes: (i) a head mounting assembly, for mounting assemblies of the system upon the head of the subject; and (ii) at least one near eye module assembly (NEMa), mounted upon the head mounting assembly, for generating optical processes or effects which act or take place upon, and are affected by, at least one eye of the subject, and for receiving results of the optical processes or effects from the at least one eye, as part of the testing, diagnosing, or treating of the vision or eyes of the subject, wherein the near eye module assembly includes: (1) a micro-display (μdisplay), for generating, and emitting, light rays which are transmitted along an incident optical path, and directed into an eye of the subject, for interacting with, and being partly reflected by, retina or/and other components of the eye; (2) a first lens assembly (L1a), for refracting the light rays generated and emitted by the micro-display into groups of parallel light rays, which are transmitted to the eye, and for refracting light rays which are reflected by the retina or/and other components of the eye; and (3) a refraction correction assembly (RCa), for correcting a wave front of the light rays paralleled by the first lens assembly, for adjusting a state of refraction of the eye, and for refracting the paralleled light rays, for regulating a state of distance perception of the eye; and (b) controlling and processing of functions, activities, and operations, of components of the head mountable unit, by a central controlling and processing unit, operatively connected to the head mountable unit.

It is to be understood that the present invention is not limited in its application to the details of type, composition, construction, arrangement, order, and number, of the system units, system sub-units, devices, assemblies, sub-assemblies, mechanisms, structures, components, elements, and configurations, and, peripheral equipment, utilities, accessories, and materials, of the system, or, or to the details of the order or sequence, number, of steps or procedures, and sub-steps or sub-procedures, of operation of the system, or of the method, set forth in the following illustrative description, accompanying drawings, and examples, unless otherwise specifically stated herein. Accordingly, the present invention is capable of other embodiments and of being practiced or carried out in various ways.

Although system units, system sub-units, devices, assemblies, sub-assemblies, mechanisms, structures, components, elements, and configurations, and, peripheral equipment, utilities, accessories, and materials, and, steps or procedures, sub-steps or sub-procedures, which are equivalent or similar to those illustratively described herein can be used for practicing or testing the present invention, suitable system units, system sub-units, devices, assemblies, sub-assemblies, mechanisms, structures, components, elements, and configurations, and, peripheral equipment, utilities, accessories, and materials, and steps or procedures, sub-steps or sub-procedures, are illustratively described and exemplified herein.

It is also to be understood that all technical and scientific words, terms, or/and phrases, used herein throughout the present disclosure have either the identical or similar meaning as commonly understood by one of ordinary skill in the art to which this invention belongs, unless otherwise specifically defined or stated herein. Phraseology, terminology, and, notation, employed herein throughout the present disclosure are for the purpose of description and should not be regarded as limiting.

It is to be fully understood that, unless specifically stated otherwise, the phrase ‘operatively connected’ is generally used herein, and equivalently refers to the corresponding synonymous phrases ‘operatively joined’, and ‘operatively attached’, where the operative connection, operative joint, or operative attachment, is according to a physical, or/and electrical, or/and electronic, or/and mechanical, or/and electro-mechanical, manner or nature, involving various types and kinds of hardware or/and software equipment and components. Additionally, it is to be fully understood that, unless specifically stated otherwise, the terms ‘connectable’, ‘connected’, and ‘connecting’, are generally used herein, and also may refer to the corresponding synonymous terms ‘joinable’, ‘joined’, and ‘joining’, as well as ‘attachable’, ‘attached’, and ‘attaching’.

Moreover, all technical and scientific words, terms, or/and phrases, introduced, defined, described, or/and exemplified, in the above Background section, are equally or similarly applicable in the illustrative description of the preferred embodiments, examples, and appended claims, of the present invention. Additionally, as used herein, the term ‘about’ refers to ±10% of the associated value.

System units, system sub-units, devices, assemblies, sub-assemblies, mechanisms, structures, components, elements, and configurations, and, peripheral equipment, utilities, accessories, and materials, steps or procedures, sub-steps or sub-procedures, as well as operation, and implementation, of exemplary preferred embodiments, alternative preferred embodiments, specific configurations, and, additional and optional aspects, characteristics, or features, thereof, of a multi-functional optometric-ophthalmic system for testing, diagnosing, or treating, vision or eyes of a subject, and methodologies thereof, according to the present invention, are better understood with reference to the following illustrative description and accompanying drawings. Throughout the following illustrative description and accompanying drawings, same reference numbers, letters, terms, or phrases, refer to same system units, system sub-units, devices, assemblies, sub-assemblies, mechanisms, structures, components, elements, and configurations, and, peripheral equipment, utilities, accessories, and materials. In selected accompanying drawings a reference XYZ coordinate axis system is shown for indicating x, y, and z, directions relative to the components drawn therein.

In the following illustrative description of the present invention, included are main or principal system units, system sub-units, devices, assemblies, sub-assemblies, mechanisms, structures, components, elements, and configurations, and, peripheral equipment, utilities, accessories, and materials, and functions thereof, and, main or principal steps or procedures, and sub-steps or sub-procedures, needed for sufficiently understanding proper ‘enabling’ utilization and implementation of the disclosed invention. Accordingly, description of various possible preliminary, intermediate, minor, or/and optional, system units, system sub-units, devices, assemblies, sub-assemblies, mechanisms, structures, components, elements, and configurations, and, peripheral equipment, utilities, accessories, and materials, or/and functions thereof, or/and, steps or procedures, or/and sub-steps or sub-procedures, which are readily known by one of ordinary skill in the art, which are available in the prior art or/and technical literature relating to the field(s) of the present invention, are at most only briefly indicated herein.

In the following illustrative description of the present invention, there is first provided illustrative description of exemplary preferred embodiments of the structure and function of the multi-functional optometric-ophthalmic system for testing, diagnosing, or treating, vision or eyes of a subject, of the present invention. Thereafter, is provided illustrative description of exemplary preferred embodiments of steps and procedures of corresponding methodologies for testing, diagnosing, or treating vision or eyes of a subject, of the present invention, utilizing the multi-functional optometric-ophthalmic system, of the present invention.

### The Multi-Functional Optometric-Ophthalmic System

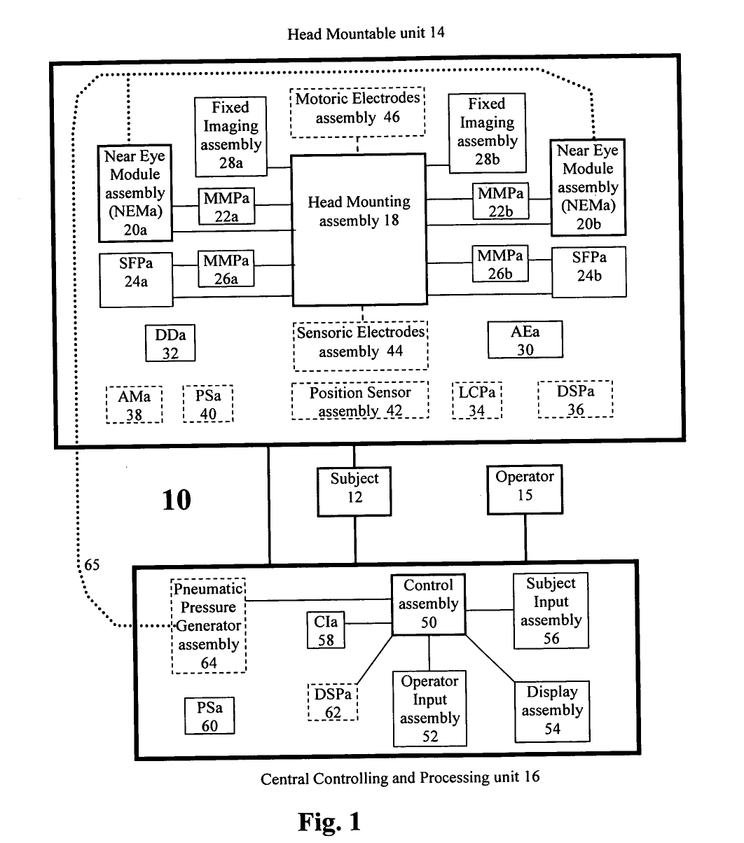

According to a main aspect of the present invention, there is provision of a multi-functional optometric-ophthalmic system for testing, diagnosing, or treating, vision or eyes of a subject. Referring now to the drawings, FIG. 1 is a block diagram illustrating an exemplary preferred embodiment of the system, herein, generally referred to as multi-functional optometric-ophthalmic system 10, and main components thereof, for testing, diagnosing, or treating, vision or eyes of a subject, herein, generally referred to as subject 12, by an operator, herein, generally referred to as operator 15. FIG. 2 is a schematic diagram illustrating an exemplary preferred embodiment of implementing multi-functional optometric-ophthalmic system 10, for testing, diagnosing, or treating, vision or eyes of subject 12, by operator 15.

As shown in FIGS. 1 and 2, multi-functional optometric-ophthalmic system 10, for testing, diagnosing, or treating, vision or eyes of a subject 12, of the present invention, includes the following main components: (a) a head mountable unit 14, and (b) a central controlling and processing unit 16. Head mountable unit 14 includes the following main components: (i) a head mounting assembly 18; and (ii) at least one near eye module assembly 20, herein, also referred to as an NEM assembly (NEMa) 20, where FIG. 1 shows head mountable unit 14 including two near eye module assemblies, i.e., near eye module assembly (NEMa) 20a and near eye module assembly (NEMa) 20b.

Head mountable unit 14, preferably, includes at least one multi-axis moving and positioning assembly 22, herein, also referred to as MMP assembly (MMP) 22, where FIG. 1 shows head mountable unit 14 including four MMP assemblies, i.e., MMP assembly (MMPa) 22a, MMP assembly (MMPa) 22b, MMP assembly (MMPa) 26a, and MMP assembly (MMPa) 26b.

Head mountable unit 14, preferably, includes at least one secondary fixation pattern assembly 24, herein, also referred to as SFP assembly (SFPa) 24, where FIG. 1 shows head mountable unit 14 including two SFP assemblies, i.e., SFP assembly (SFPa) 24a and SFP assembly (SFPa) 24b.

Head mountable unit 14, preferably, includes at least one fixed imaging assembly 28, where FIG. 1 shows head mountable unit 14 including two fixed imaging assemblies, i.e., fixed imaging assembly 28a and fixed imaging assembly 28b.

Head mountable unit 14, preferably, includes an analog electronics assembly 30, herein, also referred to as AE assembly (AEa) 30.

Head mountable unit 14, preferably, includes a display driver assembly 32, herein, also referred to as DD assembly (DDa) 32.

Head mountable unit 14, optionally, includes any number or combination of the following additional (optional) components: a local controlling and processing assembly 34, herein, also referred to as LCP assembly (LCPa) 34; a digital signal processing assembly 36, herein, also referred to as DSP assembly (DSPa) 36; an audio means assembly 38, herein, also referred to as AM assembly (AMa) 38; a power supply assembly 40, herein, also referred to as PS assembly (PSa) 40; a position sensor assembly 42; a sensoric electrodes assembly 44; and a motoric electrodes assembly 46.

Central controlling and processing unit 16, preferably, includes any number or combination of the following components: a control assembly 50; an operator input assembly 52; a display assembly 54; a subject input assembly 56; a communication interface assembly 58, herein, also referred to as CI assembly (CIa) 58; and a power supply assembly 60, herein, also referred to as PS assembly (PSa) 60.

Central controlling and processing unit 16, optionally, includes any number or combination of the following additional (optional) components: a digital signal processing assembly 62, herein, also referred to as DSP assembly (DSPa) 62; and a pneumatic pressure generator assembly 64.

In FIGS. 1 and 2, selected (i.e., not all) operative connections or linkages of electronics and communications among system components, assemblies thereof, subject 12, and operator 15, are generally indicated by a (solid) lines drawn between selected (i.e., not all) system components, assemblies thereof, subject 12, and operator 15. Exemplary operative connections or linkages include those which are shown between head mountable unit 14 and central controlling and processing unit 16; between head mountable unit 14 and subject 12; between central controlling and processing unit 16 and subject 12; and between central controlling and processing unit 16 and operator 15. Such communication connections or linkages are based on wired or/and wireless hardware, software, protocols and applications, thereof.

Additionally, for example, pneumatic pressure generator assembly 64, of central controlling and processing unit 16, is operatively connected, via a high pressure air transfer line 65, to each of the two near eye module assemblies (near eye module assembly 20a and near eye module assembly 20b). Additional exemplary operative connections are shown among selected assemblies of head mountable unit 14 and among selected assemblies of central controlling and processing unit 16. In a non-limiting manner, it is to be fully understood that, although not shown in FIG. 1, additional operative connections exist among the various assemblies of head mountable unit 14 and among the various assemblies of central controlling and processing unit 16.

Accordingly, the present invention provides various alternative exemplary preferred embodiments of a multi-functional optometric-ophthalmic system, that is, multi-functional optometric-ophthalmic system 10, for testing, diagnosing, or treating, vision or eyes of a subject.

### Head Mountable Unit

In multi-functional optometric-ophthalmic system 10, head mountable unit 14 corresponds to, and represents, an operatively integrated combination of required, preferred, and optional, assemblies (aside from those assemblies of central controlling and processing unit 16) and components thereof, which are included in a given embodiment or configuration of multi-functional optometric-ophthalmic system 10, that are used for automatically and interactively testing, diagnosing, or treating vision or eyes of subject 12, by operator 15. As illustrated in FIG. 2, for implementing multi-functional optometric-ophthalmic system 10, for testing, diagnosing, or treating, vision or eyes of subject 12, by operator 15, head mountable unit 14, including the combination of assemblies, is mounted upon the head of subject 12. As shown in FIGS. 1 and 2, head mountable unit 14 is operatively connected to central controlling and processing unit 16, and is operatively connected to (mounted upon) the head of subject 12.

Illustrative description of structure and function (operation), and selected examples thereof, of each of the required, preferred, and optional, assemblies of head mountable unit 14 in multi-functional optometric-ophthalmic system 10, follows hereinbelow.

### Head Mounting Assembly

With reference again made to FIGS. 1 and 2, in head mountable unit 14, head mounting assembly 18 is for firmly and securely mounting of the previously stated combination of required, preferred, and optional, assemblies (aside from those assemblies of central controlling and processing unit 16), which are included in a given embodiment or configuration of multi-functional optometric-ophthalmic system 10, upon the head of subject 12, in a manner such that no externally propagating light reaches, or falls upon, the volumetric region encompassing a selected portion (particularly including the eyes) of the face of subject 12 and encompassing the combination of assemblies mounted via head mounting assembly 18.

During implementation of multi-functional optometric-ophthalmic system 10, in general, and especially during operation of the combination of assemblies represented by head mountable unit 14, it is critically important that no externally propagating light reaches, or falls upon, the volumetric region encompassing a selected portion (particularly including the eyes) of the face of subject 12, or the volumetric region encompassing the combination of assemblies mounted via head mounting assembly 18. Accordingly, it is critically important that the assemblies mounted upon head mounting assembly 18 be impervious to light, or impenetrable by light, with respect to the following assemblies: near eye module assembly (NEMa) 20, multi-axis moving and positioning assembly (MMPa) 22, secondary fixation pattern assembly (SFPa) 24, fixed imaging assembly 28, local controlling and processing assembly (LCPa) 30, digital signal processing assembly (DSPa) 32, analog electronics assembly (AEa) 34, display driver assembly (DDa) 36, audio means assembly (AMa) 38, power supply assembly (PSa) 40, position sensor assembly 42, and sensoric electrodes assembly 44) mounted via head mounting assembly 18.

For performing the preceding described functions, as illustrated in FIG. 2, head mounting assembly 18 includes the main components of: (1) a light blocking sub-assembly 18a, (2) a frame sub-assembly 18b, and (3) a strap sub-assembly 18c.

Light blocking sub-assembly 18a is essentially completely imperviable (i.e., not admitting passage) to light. Light blocking sub-assembly 18a is constructed from materials such as plastics, and rubber, and similar types of synthetic or natural materials which are suitable for blocking or preventing light from impinging upon the eye region of the face of subject 12. Frame sub-assembly 18b and strap sub-assembly 18c are those components of head mounting assembly 18 upon which are mounted the various assemblies of head mountable unit 14. Frame sub-assembly 18b and strap sub-assembly 18c can be, for example, constructed or configured similar to a virtual reality type of head mountable device or apparatus, or a helmet type of head mountable device or apparatus.

Firmly and securely mounting, upon head mounting assembly 18, of the previously stated combination of required, preferred, and optional, assemblies (aside from those assemblies of central controlling and processing unit 16), which are included in a given embodiment or configuration of multi-functional optometric-ophthalmic system 10, upon the head of subject 12, is performed according to mounting techniques known in the art for mounting of miniature sized or micro-sized electrical, electronic, mechanical, electro-mechanical, optical, or/and electro-optical, components, upon a mounting assembly, such as head mounting assembly 18.

Such mounting techniques involve, for example, the use of a wide variety of different types or kinds of mounting means and mounting materials. Such mounting means and mounting materials, include, for example, holders, support elements, brackets, bars, tracks, channels, posts, nails, screws, nuts, bolts, pins, clips, clamps, connectors, joiners, adhesives, glue, cement, epoxy, tape, wires, cord, and combinations thereof, or/and similar types of assemblies, components, elements, and materials known in the art which are applicable for mounting, connecting, joining, or attaching, structures to each other.

Head mountable unit 14 is preferably designed and constructed according to appropriate geometrical (dimensional) and weight factors and parameters, such that head mountable unit 14, when mounted, via head mounting assembly 18, upon the head of subject 12 is ‘user’ friendly with respect to subject 12. In cases where subject 12 experiences discomfort due to the mounted head mountable unit 14, then, optionally, a height adjustable tripod, or an externally located supporting element, is operatively connected, via frame sub-assembly 18b of head mounting assembly 18, to head mountable unit 14.

### Near Eye Module Assembly (NEMa)

In head mountable unit 14, the at least one near eye module assembly 20, also referred to as an NEM assembly (NEMa) 20, where FIG. 1 shows head mountable unit 14 including two near eye module assemblies, i.e., near eye module assembly 20a and near eye module assembly 20b, is for generating various different types or kinds of optical processes or effects which act or take place upon, and are affected by, the eye(s) of subject 12, and for receiving the results of such optical processes or effects from the eyes, as part of the testing, diagnosing, or treating of the vision or eyes of subject 12 by multi-functional optometric-ophthalmic system 10.

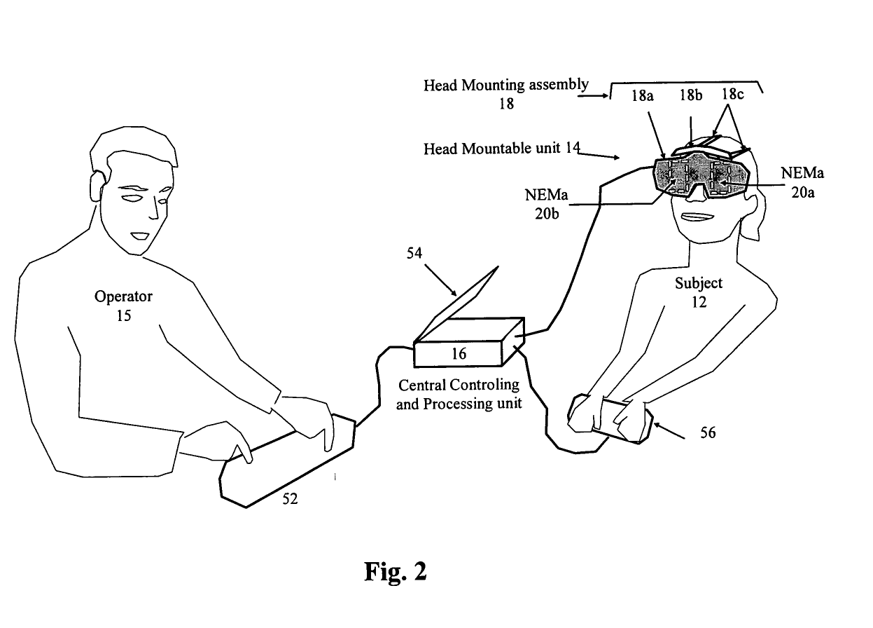

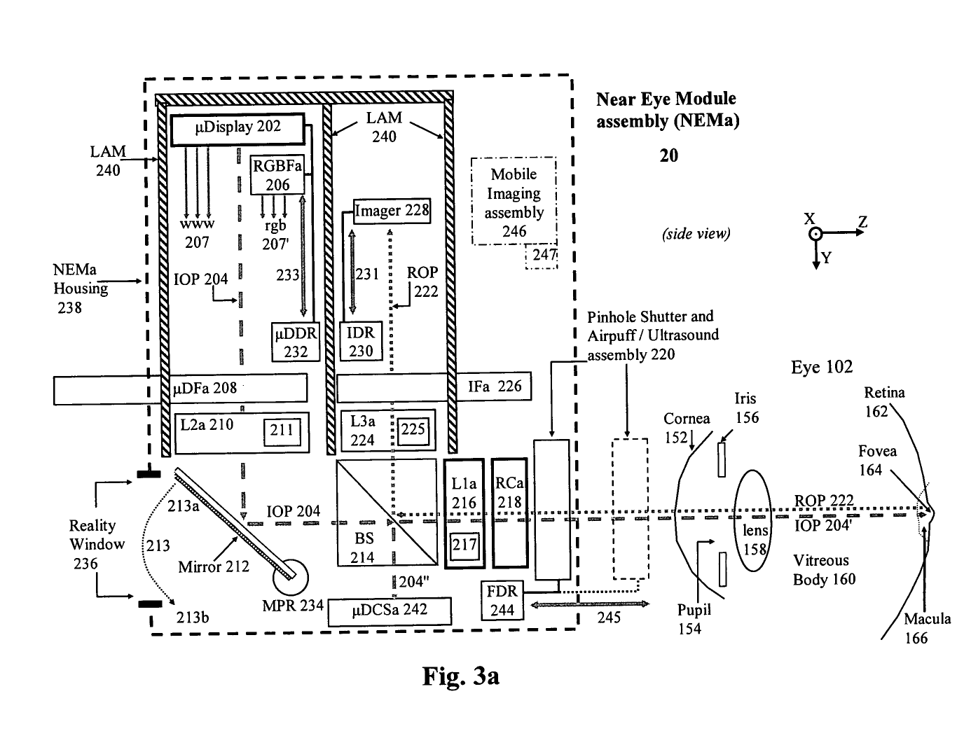

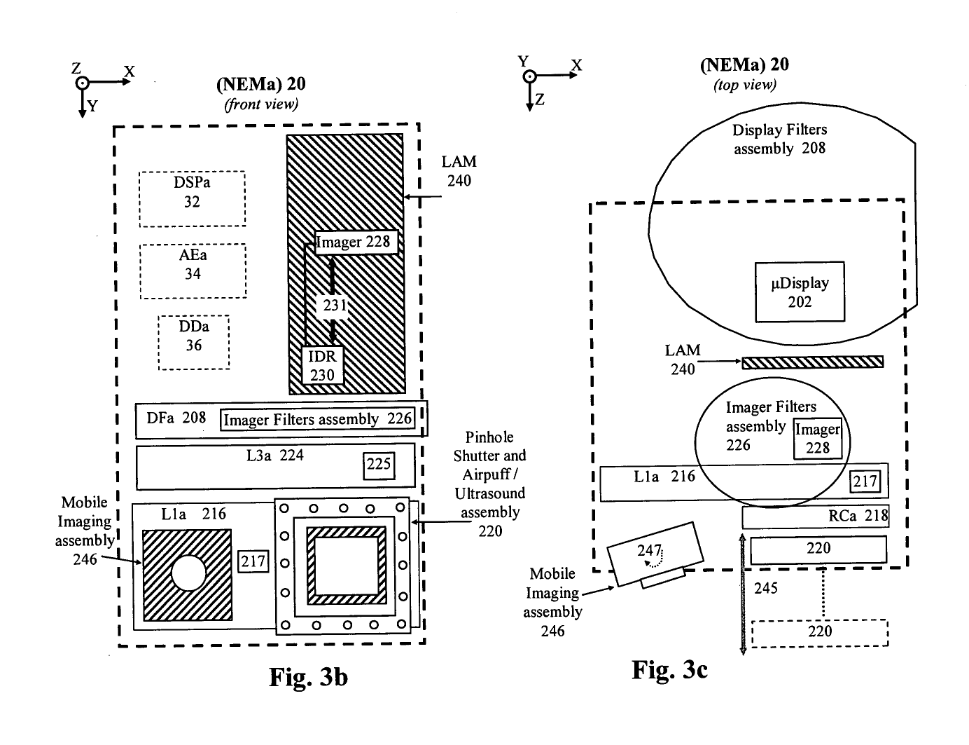

FIGS. 3a, 3b, and 3c, are schematic diagrams illustrating close-up (partly exposed) side view (FIG. 3a), front view (FIG. 3b), and top view (FIG. 3c), of an exemplary specific preferred embodiment of near eye module assembly 20 (i.e., near eye module assembly 20a or near eye module assembly 20b, of FIG. 1), and components thereof, as part of multi-functional optometric-ophthalmic system 10 illustrated in FIGS. 1 and 2.

Illustrative description of the main functions (operations) of near eye module assembly (NEMa) 20, and components thereof, with reference to FIGS. 3a, 3b, and 3c, follows. For clarity of presentation and understanding, the description is made with respect to the optical path illustrated in FIG. 3a, and generally defined by incident optical path (IOP) 204 (in FIG. 3a, indicated by ‘long’ dashed lines or ‘rectangles’), generated by micro-display (μdisplay) 202, which is directed into eye 102 of subject 12, for interacting with, and being partly reflected by, retina 162 (and possibly other components) of eye 102, for forming reflection optical path (ROP) 222 (in FIG. 3a, indicated by ‘short’ dashed lines or ‘squares’).

With reference to FIGS. 3a, 3b, and 3c, near eye module assembly (NEMa) 20 includes the main components of: (1) a micro-display (μdisplay) 202, (2) a first lens assembly (L1a) 216, and (3) a refraction correction assembly (RCa) 218.

Micro-display (μdisplay) 202 is for generating, and emitting, light rays which are transmitted along incident optical path (IOP) 204, and directed into eye 102 of subject 12, for interacting with, and being partly reflected by, retina 162 (and possibly other components) of eye 102.

A first exemplary specific preferred embodiment of the present invention is wherein micro-display (μdisplay) 202 generates, and emits, normal intensity patterns, pictures, or/and videos, which are transmitted to eye 102 of subject 12. Subject 12 reacts to the transmitted pattern, picture, or/and video, according to the properties, characteristics, and parameters, thereof. A second exemplary specific preferred embodiment of the present invention is wherein micro-display (μdisplay) 202 generates, and emits, short interval pulses (e.g., on the order of milliseconds (ms) time duration) of high intensity pattern or illumination, which are transmitted to eye 102 of subject 12. Retina 162 (and possibly other components) of eye 102 reflect(s) the transmitted high intensity pattern or illumination (via a variety of other optical components of near eye module assembly (NEMa) 20) into an imager 228 (described further hereinbelow).

Micro-display (μdisplay) 202 generates and emits white light rays having a spectrum including wavelengths in a range of, preferably, between about 200 nanometers (nm) and about 10,000 nanometers (nm), and more preferably, between about 400 nanometers (nm) and about 1000 nanometers (nm). Micro-display (μdisplay) 202 is, preferably, designed, constructed, and operates, preferably, according to organic LED (light emitting diode) technology.

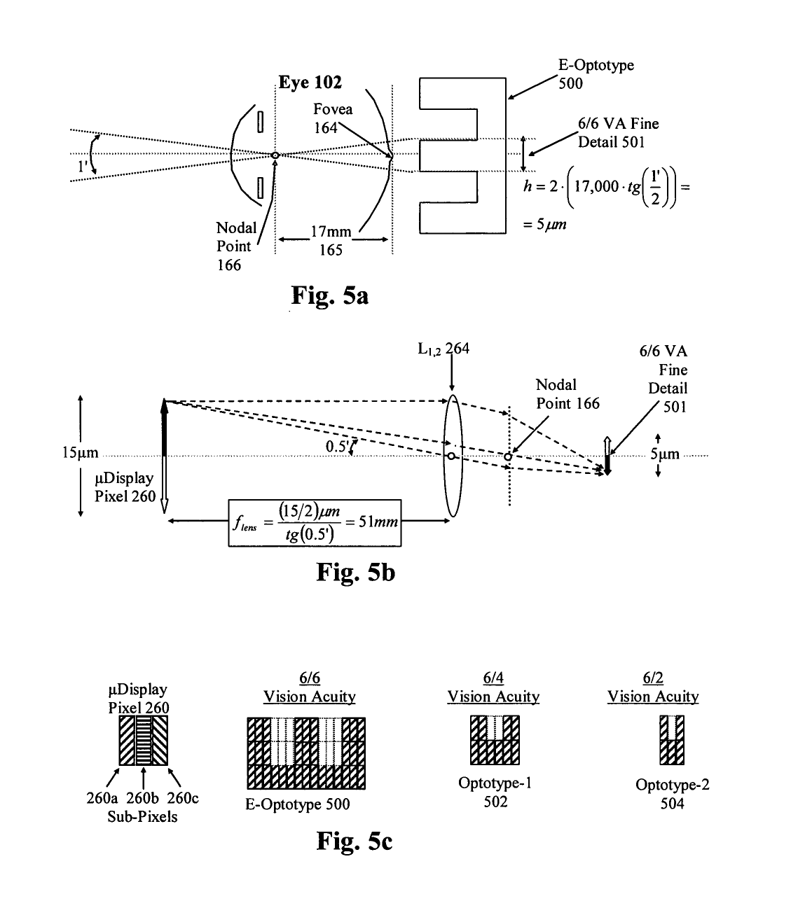

Micro-display (μdisplay) 202 has an active display area with a resolution of, preferably, 900 pixels×600 pixels, wherein pixel size is, preferably, 15 microns (μm)×15 microns (μm), and wherein each pixel is partitioned into three sub-pixels, each of size 5 microns (μm)×15 microns (μm). Such partitioning of the pixels is done for enabling conversion of white light rays (in FIG. 3a, indicated by three arrows referenced by the symbol ‘www’ and number 207) generated by, and emitted from, micro-display (μdisplay) 202, to colored light rays (in FIG. 3a, indicated by three arrows referenced by the symbol ‘rgb’ and number 207′) exiting from a tri-color filter assembly, for example, red-green-blue filter assembly (RGBFa) 206 (described further hereinbelow). Such partitioning of the pixels is also done for testing vision acuities higher than 6/6, based on the 6/6 vision acuity design requirement illustrated in FIGS. 5a and 5b (described further hereinbelow).

First lens assembly (L1a) 216 has two main functions. The first main function of first lens assembly (L1a) 216 is for refracting the light rays generated and emitted by micro-display (μdisplay) 202 into groups of parallel light rays, which are transmitted to eye 102 of subject 12. The second main function of first lens assembly (L1a) 216 is for refracting light rays which are reflected by retina 162 (or/and other components, for example, cornea 152) of eye 102 of subject 12. Preferably, light rays correspond to the eye reflections of the normal intensity patterns, pictures, or/and videos, or, of the high intensity pattern or illumination, generated and emitted by micro-display (μdisplay) 202, as previously described hereinabove.

First lens assembly (L1a) 216, preferably, includes an in/out moving and positioning sub-assembly, for example, in/out moving and positioning sub-assembly 217, which enables moving and positioning of first lens assembly (L1a) 216 in or out of incident optical path (IOP) 204 directed into eye 102, according to a particular mode of operation of near eye module assembly (NEMa) 20. In/out moving and positioning sub-assembly 217, is, for example, a solenoid which is operatively connected to the components of first lens assembly (L1a) 216.

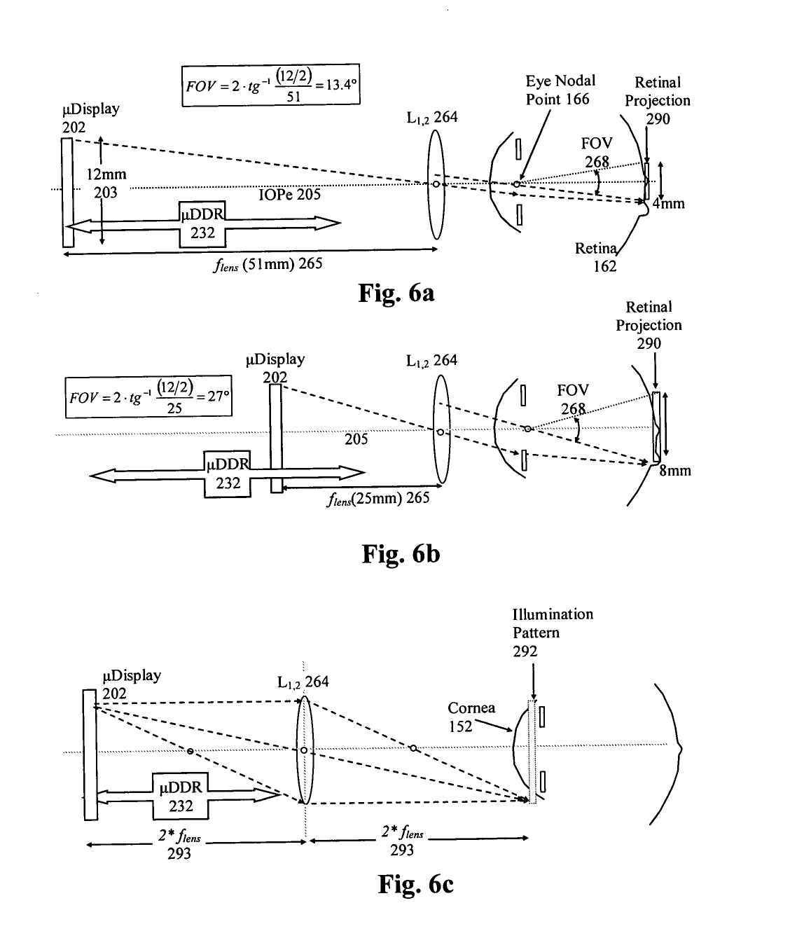

Additional detailed illustrative description of utilizing first lens assembly (L1a) 216 is provided hereinbelow, in the sub-section ‘Special Design Requirements and Characteristics of the Near Eye Module Assembly’, along with reference to FIGS. 6a, 6b, and 6c.

Refraction correction assembly (RCa) 218 has two main functions. The first main function of refraction correction assembly (RCa) 218 is for correcting the wave front of the light rays that are paralleled by first lens assembly (L1a) 216, for the purpose of adjusting the state of refraction of eye 102 of subject 12. The second main function of refraction correction assembly (RCa) 218 is for refracting the light rays that are paralleled by first lens assembly (L1a) 216, for the purpose of regulating the state of distance perception of eye 102 of subject 12. For performing the preceding main functions, refraction correction assembly (RCa) 218 includes components and functionalities thereof, according to a spherical type correction, a cylindrical type correction, a prismatic type correction, or a combination thereof.

According to a spherical type correction, refraction correction assembly (RCa) 218 includes components and functionalities thereof, for correcting (via compensating) a myopic or hyperopic refractive condition of eye 102 of subject 12, or/and for emulating distance of perception by subject 12 of a virtual object displayed by micro-display (μdisplay) 202. In an exemplary specific embodiment of a spherical type correction, refraction correction assembly (RCa) 218, preferably, includes a variable spherical power lens. The variable spherical power lens can be of a variable ‘liquid’ type spherical power lens, for example, as taught in the disclosures [2, 3] of Berge et al. Alternatively, the variable spherical power lens can be of a variable ‘mechanical’ type spherical power lens, for example, an Alvarez lens, for example, as taught by Schweigerling, J. [1].

In an alternative exemplary specific embodiment of operating near eye module assembly (NEMa) 20 for performing the hereinabove described main functions of refraction correction assembly (RCa) 218, according to a spherical type correction, there is changing (via decreasing or increasing) the optical distance extending between micro-display (μdisplay) 202 and first lens assembly (L1a) 216, along incident optical path (IOP) 204 directed into eye 102, (in FIG. 3a, indicated by bi-directional arrow 233), according to any of the three modes illustratively described hereinbelow regarding description of the structure/function of micro-display distance regulator (μDDR) 232.

According to a cylindrical type correction, refraction correction assembly (RCa) 218 includes components and functionalities thereof, for correcting (via compensating) an astigmatic condition of eye 102 of subject 12. In an exemplary specific embodiment of a cylindrical type correction, refraction correction assembly (RCa) 218, preferably, includes a variable cylindrical power lens having a selectable axis. The variable cylindrical power lens can be of a variable ‘mechanical’ type cylindrical power lens, for example, a Humphrey lens, for example, as taught by Schweigerling, J. [1]. In particular, wherein the cylindrical power and the axis of the Humphrey lens are selected by translating the two plates thereof in opposite directions.

According to a prismatic type correction, refraction correction assembly (RCa) 218 includes components and functionalities thereof, for correcting (via compensating) binocular alignment errors (e.g., strabismus) of a pair of eyes 102. In an exemplary specific embodiment of a prismatic type correction, refraction correction assembly (RCa) 218, preferably, includes a variable prismatic power lens having a selectable axis. The variable prismatic power lens can be a Risley prism, for example, as taught by Schweigerling, J. [1]. In particular, wherein the axis of the Risley prism is selected by rotating of the entire Risley prism structure of two counter-rotating wedge prisms whose bases are in opposite directions.

According to a combination of a spherical type correction and a cylindrical type correction, refraction correction assembly (RCa) 218 includes components and functionalities thereof, and operates in a manner, based on a combination of the preceding illustratively described spherical type correction, or/and cylindrical type correction, or/and prismatic type correction.

A third function of refraction correction assembly (RCa) 218 is for regulating monocular distance perception of virtual objects perceived by subject 12 of a virtual object displayed by micro-display (μdisplay) 202, as illustratively described hereinbelow, in the procedure ‘Monocular Distance Perception Regulation’.

Referring again to FIGS. 3a, 3b, and 3c, near eye module assembly (NEMa) 20, preferably, includes any number and combination of the following additional components: a red-green-blue filter assembly (RGBFa) 206, a micro-display filters assembly (μDFa) 208, a second lens assembly (L2a) 210, a mirror 212, a beam splitter 214, a pinhole shutter and airpuff/ultrasound assembly 220, a third lens assembly (L3a) 224, imager filters assembly 226, imager 228, imager distance regulator (IDR) 230, micro-display distance regulator (μDDR) 232, mirror position regulator (MPR) 234, a reality window 236, an NEMa housing 238, a light absorbing material (LAM) 240, a micro-display calibration sensor assembly (μDCSa) 242, and frontal distance regulator (FDR) 244, and a mobile imaging assembly 246.

Red-green-blue filter assembly (RGBFa) 206 is for converting white light rays (in FIG. 3a, arrows ‘www’ 207), generated by, and emitted from, micro-display (μdisplay) 202, to colored light rays (in FIG. 3a, arrows ‘rgb’ 207′) which travel along incident optical path (IOP) 204 directed into eye 102. Red-green-blue filter assembly (RGBFa) 206 is of a configuration, preferably, designed, constructed, and operative, physically adjacent to micro-display (μdisplay) 202, in a manner such that red-green-blue filter assembly (RGBFa) 206 covers only a small part (corresponding to a size, preferably, of about 10%, corresponding to about 90 pixels×600 pixels) of the total active display area having a resolution of, preferably, 900 pixels×600 pixels. Such a configuration enables micro-display (μdisplay) 202 to simultaneously generate and emit white light rays (‘www’ 207) via an unfiltered zone of micro-display (μdisplay) 202, and colored light rays (‘rgb’ 207′) via a filtered zone of micro-display (μdisplay) 202.

Micro-display filters assembly (μDFa) 208 is for selectively filtering the preceding illustratively described filtered or/and non-filtered parts of the light rays generated and emitted by micro-display (μdisplay) 202. Micro-display filters assembly (μDFa) 208 is, preferably, a collection of filter windows configured in a form of a rotatable wheel. The filter windows are, preferably, a band-pass type, having a band pass of about 50 nanometers (nm). The collection of filter windows enables selecting wavelengths in a range of, preferably, between about 200 nanometers (nm) and about 10,000 nanometers (nm), and more preferably, between about 400 nanometers (nm) and about 1000 nanometers (nm). In general, the filter windows are of any type, for example, colored filter windows or/and interference filters. Such a rotatable wheel, preferably, includes a transparent filter window that is transparent to light, for the optional mode of operation wherein the incident light rays passing through are non-filtered.

Second lens assembly (L2a) 210 is for increasing optical power over that provided by first lens assembly (L1a) 216. Second lens assembly (L2a) 210 is used for increasing the optical power over that provided by first lens assembly (L1a) 216 when the optical distance extending between micro-display (μdisplay) 202 and first lens assembly (L1a) 216, along incident optical path (IOP) 204 directed into eye 102, is decreased as a result of an increase in the field of view generated by micro-display (μdisplay) 202.

Second lens assembly (L2a) 210, preferably, includes an in/out moving and positioning sub-assembly, for example, in/out moving and positioning sub-assembly 211, which enables moving and positioning of second lens assembly (L1a) 210 in or out of incident optical path (IOP) 204 directed into eye 102, according to a particular mode of operation of near eye module assembly (NEMa) 20. In/out moving and positioning sub-assembly 211, is, for example, a solenoid which is operatively connected to the components of second lens assembly (L2a) 210.

Mirror 212 has two main functions. The first main function of mirror 212 is for changing the direction of the light rays generated and emitted by micro-display (μdisplay) 202. Such direction change of the generated and emitted light rays, thereby, partly defines the incident optical path (IOP) 204 extending between micro-display (μdisplay) 202 and eye 102 of subject 12. The second main function of mirror 212 is for serving as a controllable ‘gate’ or barrier, for controllably gating or blocking eye 102 of subject 12 from being exposed to the local environment external to, and outside of, near eye module assembly (NEMa) 20.

Mirror 212 is, preferably, operatively connected to mirror position regulator (MPR) 234, which is actuated and operative for regulating or changing the position of mirror 212, in particular, along mirror positioning arc 213 spanning between a first mirror position 213a and a second mirror position 213b. Such an embodiment of near eye module assembly (NEMa) 20 is for opening a reality window 236, for the purpose of exposing eye 102 of subject 12 to the environment beyond reality window 236 of near eye module assembly (NEMa) 20.

Beam splitter 214 is for splitting the light rays generated and emitted by micro-display (μdisplay) 202 into two groups of light rays. The first group of light rays passes through beam splitter 214 and continues along incident optical path (IOP) 204′ and into eye 102 of subject 12, for interacting with, and being partly reflected by, retina 162 (and possibly other components) of eye 102. The second group of light rays reflects off beam splitter 214 and continues along incident optical path (IOP) 204″ and into micro-display calibration sensor assembly (μDCSa) 242. In general, beam splitter 214 is any type of beam splitter optical element, and is, preferably, a beam splitter characterized by a 50% transmission of light rays.

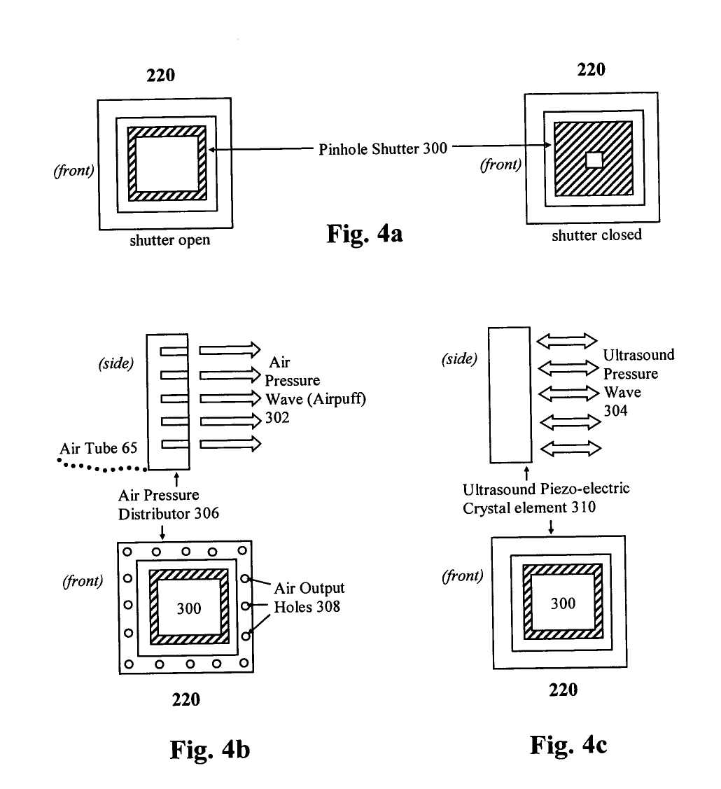

Pinhole shutter and airpuff/ultrasound assembly 220 has two main functions. The main functions, and components, of pinhole shutter and airpuff/ultrasound assembly 220 are illustratively described herein as follows, with reference to FIGS. 4a, 4b, and 4c, being schematic diagrams illustrating front and side views of different exemplary specific preferred embodiments of pinhole shutter and airpuff/ultrasound assembly 220, and components thereof, as part of near eye module assembly (NEMa) (20 in FIGS. 3a, 3b, and 3c; 20a or 20b, in FIG. 1), of multi-functional optometric-ophthalmic system 10 illustrated in FIGS. 1 and 2.

The first main function of pinhole shutter and airpuff/ultrasound assembly 220 is for controlling intensity of the first group of light rays which exits beam splitter 214 and continues along incident optical path (IOP) 204′ into eye 102 of subject 12. For this function, pinhole shutter and airpuff/ultrasound assembly 220 includes a pinhole type shutter, for example, pinhole shutter 300 (FIGS. 4a, 4b, 4c), having a variable sized aperture with a shutter open configuration (FIG. 4a, left side), and a shutter closed configuration (FIG. 4b, right side) configuration.

The second main function of pinhole shutter and airpuff/ultrasound assembly 220 is for applying an air pressure wave, for example, air pressure wave (airpuff) 302 (FIG. 4b), or, alternatively, for applying an ultrasound wave, for example, ultrasound pressure wave 304 (FIG. 4c), onto cornea 152 (FIG. 3a) of eye 102 of subject 12.

For applying air pressure wave (airpuff) 302 onto cornea 152 of eye 102, pinhole shutter and airpuff/ultrasound assembly 220 includes an air pressure distributor, for example, air pressure distributor 306 (FIG. 4b), having air output holes 308, for distributing air pressure wave (airpuff) 302 generated by, and received (via high pressure air transfer line 65) from, pneumatic pressure generator assembly 64 (FIG. 1) of central controlling and processing unit 16, to cornea 152 of eye 102 of subject 12. Response by cornea 152 to the applied air pressure wave (airpuff) 302 is sensed and received by imager 228 of near eye module assembly (NEMa) 20, or/and by fixed imaging assembly 28 of head mountable unit 14, or/and by mobile imaging assembly 246 of near eye module assembly (NEMa) 20.

For applying ultrasound pressure wave 304 onto cornea 152 of eye 102, pinhole shutter and airpuff/ultrasound assembly 220 includes an ultrasound wave transducer 310 (FIG. 4c), for generating and distributing ultrasound pressure wave 304 to cornea 152 of eye 102 of subject 12. Ultrasound wave transducer 310 is, preferably, an ultrasound piezo-electrical crystal element 310. Response by cornea 152 to applied ultrasound pressure wave 304 is sensed and received, preferably, by ultrasound wave transducer 310 (in FIG. 4c, indicated by the two directional arrows of ultrasound pressure wave 304).

Third lens assembly (L3a) 224 is for increasing optical power over that provided by first lens assembly (L1a) 216. Third lens assembly (L3a) 224 is used for increasing the optical power over that provided by first lens assembly (L1a) 216 when the optical distance extending between imager 228 and first lens assembly (L1a) 216, along reflection optical path (ROP) 222 directed out of eye 102, is decreased as a result of an increase in the field of view (via a decrease in imaging resolution) which is sensed by imager 228.

Third lens assembly (L3a) 224, preferably, includes an in/out moving and positioning sub-assembly, for example, in/out moving and positioning sub-assembly 225, which enables moving and positioning of third lens assembly (L3a) 224 in or out of reflection optical path (ROP) 222, according to a particular mode of operation of near eye module assembly (NEMa) 20. In/out moving and positioning sub-assembly 225, is, for example, a solenoid which is operatively connected to the components of third lens assembly (L3a) 224.

Imager filters assembly 226 is for selectively filtering light rays reflected by retina 162 (or/and other components, for example, cornea 152) of eye 102 of subject 12, which pass through the various optical components, for example, refraction correction assembly (RCa) 218, first lens assembly (L1a) 216, beam splitter 214, and third lens assembly (L3a) 224, along reflection optical path (ROP) 222, en route to imager 228.

Imager filters assembly 226 is, preferably, a collection of filter windows configured in a form of a rotatable wheel. The filter windows are, preferably, a band-pass type, having a band pass of about 50 nanometers (nm). The collection of filter windows enables selecting wavelengths in a range of, preferably, between about 200 nanometers (nm) and about 10,000 nanometers (nm), and more preferably, between about 400 nanometers (nm) and about 1000 nanometers (nm). In general, the filter windows are of any type, for example, colored filter windows or/and interference filters. Such a rotatable wheel, preferably, includes a transparent filter window that is transparent to light, for the optional mode of operation wherein the reflected light rays passing through are non-filtered.

Imager 228 is for capturing still or video patterns or images which are reflected by retina 162 (or/and other components, for example, cornea 152) of eye 102 of subject 12. Imager 228 is, preferably, designed, constructed, and operates, preferably, according to complementary methyl-oxide semiconductor (CMOS) image sensor technology, or, alternatively, according to charged coupled detector (CCD) technology, or, alternatively, according to technologies sufficiently sensitive for detecting ultra-violet (UV) or infra-red (IR) spectra.

Imager 228 has an active sensing area with a resolution of, preferably, 1600 pixels×1200 pixels, wherein pixel size is, preferably, 3 microns (μm)×3 microns (μm). Imager 228 senses light rays having a spectrum including wavelengths in a range of, preferably, between about 200 nanometers (nm) and about 10000 nanometers (nm), and more preferably, between about 400 nanometers (nm) and about 1000 nanometers (nm).

Imager distance regulator (IDR) 230 is for regulating or changing (via decreasing or increasing) the optical distance extending between first lens assembly (L1a) 216 and imager 228, along reflection optical path (ROP) 222 directed out of eye 102. Regulating or changing of this optical distance (in FIGS. 3a and 3b, indicated by bi-directional arrow 231) is done for two main reasons: (1) to adjust and attain a fine focus of the reflected light rays impinging upon imager 228, and (2) to match the focal distance corresponding to the optical power provided by first lens assembly (L1a) 216, and when applicable, third lens assembly (L3a) 224, along reflection optical path (ROP) 222.

Micro-display distance regulator (μDDR) 232 is for regulating or changing (via decreasing or increasing) the optical distance extending between micro-display (μdisplay) 202 and first lens assembly (L1a) 216, along incident optical path (IOP) 204 directed into eye 102. Regulating or changing of this optical distance (in FIG. 3a, indicated by bi-directional arrow 233) is performed for four main reasons: (1) to match the focal distance corresponding to the optical power provided by first lens assembly (L1a) 216 and second lens assembly (L2a) 210, along incident optical path (IOP) 204, or (2) to correct (via compensating) a myopic or hyperopic refractive condition of eye 102 of subject 12, or (3) to emulate distance of perception by subject 12 of a virtual object displayed by micro-display (μdisplay) 202, or (4) to adjust and attain a fine focal distance of light rays passing through a filter assembly, in particular, micro-display filters assembly (μDFa) 208, according to those wavelengths of light rays which are not filtered by micro-display filters assembly (μDFa) 208, or, a combination of main reasons (1)-(4).

The preceding described main function of micro-display distance regulator (μDDR) 232 is performed according to any of the following three modes:

In a first mode, there is (forward or backward) moving of micro-display (μdisplay) 202 (e.g., via micro-display distance regulator (μDDR) 232) along incident optical path (IOP) 204, relative to first lens assembly (L1a) 216 being maintained stationary at a fixed position along incident optical path (IOP) 204.

In a second mode, there is (forward or backward) moving of first lens assembly (L1a) 216 (e.g., via a distance regulator) along incident optical path (IOP) 204, relative to micro-display (μdisplay) 202 maintained stationary at a fixed position along incident optical path (IOP) 204.

In a third mode, there is (forward or backward) moving of micro-display (μdisplay) 202 (e.g., via micro-display distance regulator (μDDR) 232) along incident optical path (IOP) 204, relative to (forward or backward) moving of first lens assembly (L1a) 216 (e.g., via a distance regulator) along incident optical path (IOP) 204.

Mirror position regulator (MPR) 234 is for regulating or changing the position of mirror 212, in particular, along mirror positioning arc 213 spanning between a fully open mirror position 213a and a fully closed (or shut) mirror position 213b. Such an embodiment of near eye module assembly (NEMa) 20 is for opening a reality window 236, for the purpose of exposing eye 102 of subject 12 to the environment beyond reality window 236 of near eye module assembly (NEMa) 20. This corresponds to the second main function of mirror 212, as illustratively described hereinabove, for serving as a controllable ‘gate’ or barrier, for controllably gating or blocking eye 102 of subject 12 from being exposed to the local environment beyond reality window 236 of near eye module assembly (NEMa) 20. Mirror position regulator (MPR) 234 is, for example, a stepper type motor, or a rotational actuator, which is operatively connected to the components of mirror 212.

Reality window 236 is for exposing eye 102 of subject 12 to the ‘real’ environment external to, and outside of, near eye module assembly (NEMa) 20. Reality window 236 is used for those specific embodiments of near eye module assembly (NEMa) 20 wherein first lens assembly (L1a) 216 is not included along incident optical path (IOP) 204, and wherein mirror 212 is in a fully open mirror position 213b. When eye 102 of subject 12 is exposed to reality window 236, refraction correction assembly (RCa) 218, which is included along incident optical path (IOP) 204, functions by adjusting the state of refraction of eye 102 of subject 12.

NEMa housing 238 is for housing or ‘physically’ encompassing (containing or bounding) the various components (i.e., assemblies, sub-assemblies, etc.) of near eye module assembly (NEMa) 20. In general, any number and combination of components of near eye module assembly (NEMa) 20 are physically connected to or/and mounted on a NEMa housing 238 structure.

Light absorbing material (LAM) 240 is for absorbing stray light which is generated by micro-display (μdisplay) 202, whose presence along the optical paths of near eye module assembly (NEMa) 20, is undesirable, and which may interfere with operation and functionality of imager 228 of near eye module assembly (NEMa) 20, as well as possibly interfering with functionality of eye 102 of subject 12. Light absorbing material (LAM) 240 is configured, preferably, wherever physically possible, as part of, inside of, and among the other components of, near eye module assembly (NEMa) 20, in a manner such that light absorbing material (LAM) 240 does not obscure, block, or interfere with, the various optical paths, in particular, incident optical path (IOP) 204, incident optical path (IOP) 204′, incident optical path (IOP) 204″, and reflection optical path (ROP) 222, present within near eye module assembly (NEMa) 20.

Micro-display calibration sensor assembly (μDCSa) 242 has two main functions. The first main function of micro-display calibration sensor assembly (μDCSa) 242 is for measuring, and testing, emission power of micro-display (μdisplay) 202, which eventually decreases during normal operation of micro-display (μdisplay) 202. The second main function of micro-display calibration sensor assembly (μDCSa) 242 is for safety purposes, namely, for measuring, and according to pre-determined operating conditions criteria, for deactivating micro-display (μdisplay) 202. Exemplary operating condition criteria are hardware or/and software malfunctions of micro-display (μdisplay) 202 which cause micro-display (μdisplay) 202 to emit light rays having excess intensity or/and excessive time periods of illumination which are hazardous to eye 102 of subject 12.

Frontal distance regulator (FDR) 244 is for regulating or changing (via decreasing or increasing) the optical distance extending between pinhole shutter and airpuff/ultrasound assembly 220 and eye 102 (particularly, a foremost point on the outer surface of cornea 152 of eye 102), along incident optical path (IOP) 204′ directed into eye 102. Regulating or changing of this optical distance (in FIGS. 3a and 3c, indicated by bi-directional arrow 245) is done for two main reasons: (1) to enable placing pinhole shutter 300 (FIGS. 4a, 4b, 4c) of pinhole shutter and airpuff/ultrasound assembly 220 at a position as close as possible in front of a foremost point on the outer surface of cornea 152, for controlling intensity of the first group of light rays which exits beam splitter 214 and continues along incident optical path (IOP) 204′ into eye 102 of subject 12, and (2) to enable placing pressure distributor 306 (FIG. 4b), or ultrasound piezo-electrical crystal element 310, at an appropriate position (distance) in front of a foremost point on the outer surface of cornea 152, according to pinhole shutter and airpuff/ultrasound assembly 220 applying an air pressure wave, for example, airpuff wave 302 (FIG. 4b), or, alternatively, applying an ultrasound wave, for example, ultrasound wave 304, onto cornea 152 of eye 102 of subject 12, respectively.

Mobile imaging assembly 246 is for imaging anterior parts of eye 102, in particular, and for imaging facial anatomical features and characteristics in the immediate region of eye 102 of subject 12. As the name of mobile imaging assembly 246 implies, mobile imaging assembly 246 is ‘mobile’ relative to eye 102, by way of being included inside of near eye module assembly (NEMa) 20, which is a ‘mobile’ component of head mountable unit 14. Mobile imaging assembly 246 includes the main components of: (1) a multi-spectral illumination source, (2) an imager, and (3) an electronically adjustable focus lens. Mobile imaging assembly 246, preferably, includes a tilt angle regulator (TAR) 247.

The multi-spectral illumination source is used for selectively generating and transmitting light rays having a spectrum including wavelengths in a range of, preferably, between about 200 nanometers (nm) and about 10,000 nanometers (nm), and more preferably, between about 400 nanometers (nm) and about 1000 nanometers (nm). The multi-spectral illumination source includes, preferably, a configuration of LEDs (light emitting diodes) exhibiting a variety of different spectral properties and characteristics.

The imager is for sensing light rays having the same spectrum as indicated above. The imager includes the capability of operating at a frame rate above about 200 frames per second. The electronically adjustable focus lens is designed, constructed, and operative, for achieving a correspondence with the distance between imager of mobile imaging assembly 246 and a facial anatomical feature or characteristic in the immediate region of eye 102 of subject 12. Such correspondence occurs when sharply focused images of iris 156 and pupil 154 of eye 102 are sensed by the imager.

Tilt angle regulator (TAR) 247 is for regulating or changing the angle by which mobile imaging assembly 246 is titled relative to the front region of near eye module assembly (NEMa) 20, for example, as shown in FIG. 3c.

According to the preceding described main function and structure, mobile imaging assembly 246 has a variety of several different uses or applications as part of overall operation of near eye module assembly (NEMa) 20, each of which is illustratively described as follows.

The first main use or application of mobile imaging assembly 246 is for capturing or collecting information and data for the purpose of mapping facial anatomical features and characteristics in the immediate region of eye 102 of subject 12.

The second main use or application of mobile imaging assembly 246 is for determining distance, and determining alignment status, of a position or location of near eye module assembly (NEMa) 20 relative to eye 102 of subject 12.

The third main use or application of mobile imaging assembly 246 is for tracking positions, motion, and geometry, of pupil 154 of eye 102.

The fourth main use or application of mobile imaging assembly 246 is for observing and measuring changes in facial anatomical features or characteristics in the immediate region of eye 102 of subject 12.

The fifth main use or application of mobile imaging assembly 246 is for observing and measuring occurrence, and rate, of winking or blinking of eye 102 of subject 12.

The sixth main use or application of mobile imaging assembly 246 is for observing and measuring occurrence, properties, and characteristics, of tearing of eye 102 of subject 12.

The seventh main use or application of mobile imaging assembly 246 is for measuring and mapping thickness and topography of cornea 152 of eye 102 of subject 12.

### Special Design Requirements and Characteristics of the Near Eye Module Assembly

Hereinbelow are illustratively described special design requirements and characteristics of near eye module assembly (NEMa) (20 in FIGS. 3a, 3b, and 3c; 20a or 20b, in FIG. 1), of the multi-functional optometric-ophthalmic system (10 illustrated in FIGS. 1 and 2), in particular, regarding: (1) requirement for 6/6 vision acuity (VA), (2) increasing (expanding, widening) of the field of view (FOV), and (3) imaging of cornea 152 of eye 102.

### 6/6 Vision Acuity (VA) Requirement

Reference is made to FIG. 5a, a schematic diagram illustrating an optical diagram showing an exemplary calculation of size dimension, h, of fine detail projected onto a fovea of an eye, corresponding to 1′ angle of view, regarding the 6/6 vision acuity (VA) design requirement of the near eye module assembly (NEMa) (20 in FIGS. 3a, 3b, and 3c; 20a or 20b, in FIG. 1), of the multi-functional optometric-ophthalmic system (10 illustrated in FIGS. 1 and 2).

The definition of 6/6 vision is the ability to resolve a spatial pattern which is separated by 1′ (one minute of arc), for example, as shown in FIG. 5a. Using a simplified optical model of eye 102, one assumes that the distance, L_{NP2F}, between nodal point 166 and the fovea 164, is about 17 mm. Therefore, the ability of eye 102 to distinguish object detail specifically of 1′ corresponds to 6/6 VA Fine Detail 501 of the E-Optotype 500 image. Size dimension, h, of 6/6 VA Fine Detail 501 on the fovea 164, is calculated to be 5 microns (μm).

Based on the preceding calculated size dimension, h, the optical configuration of near eye module assembly (NEMa) 20 is to be designed, constructed, and operated, for projecting a spatial pattern of at least about 5 μm onto fovea 164. The typical size of a pixel 260 of micro-display (μdisplay) 202 is about 15 μm. Therefore, in order to project 6/6 VA Fine Detail 501 on fovea 164, the focal distance, fₗₑₙₛ, of first lens assembly (L1a) 216 used with micro-display (μdisplay) 202 is calculated to be 51 mm, as shown in FIG. 5b. In FIG. 5b, an ‘effective’ lens, L_{1,2}, 264, corresponding to an optical configuration including first lens assembly (L1a) 216, singly, or, optionally, in combination with second lens assembly (L2a) 210, is used for indicating generality of the optical configuration, while at the same time, preserving clarity of the subject matter illustratively described therein.

FIG. 5c is a schematic diagram illustrating different exemplary specific embodiments or configurations of optotypes (generated by micro-display (μdisplay) 202), used for testing vision acuities higher than 6/6, based on the 6/6 vision acuity design requirement illustrated in FIGS. 5a and 5b. Vision acuities higher than 6/6 can be tested using the different exemplary specific embodiments or configurations of optotypes generated by micro-display (μdisplay) 202. As shown in FIG. 5c, each pixel of micro-display (μdisplay) 202 consists of three sub-pixels (260a, 260b, and 260c), for example, each of size 5 microns (μm)×15 microns (μm), with vertical orientation. Therefore, for vision acuity that is higher than 6/6 (i.e., E-optotype 500), other test patterns are derived by using various combinations of sub-pixels, whereby such test patterns are used for performing the tests of higher vision acuities. For example, as shown in FIG. 5c, for performing 6/4 vision acuity or 6/2 vision acuity tests, there is using test patterns of Optotype-1 502, or Optotype-2 504, respectively.

FIG. 6a is a schematic diagram illustrating a calculation of the field of view (FOV), based on the 6/6 vision acuity design requirement illustrated in FIGS. 5a and 5b. The optical diagram schematically illustrated in FIG. 6a shows an exemplary preferred embodiment of an ‘effective’ incident optical path (IOPe) 205 extending between micro-display (μdisplay) 202 and eye 102 of subject 12, along which is an operative configuration of selected components of the NEMa, which characterizes the field of view generated by the micro-display (μdisplay) 202.

Field of view (FOV) 268 is readily calculated from the preceding illustratively described 6/6 vision acuity requirement, as follows. For micro-display (μdisplay) 202 having been moved and positioned (via micro-display distance regulator (μDDR) 232), and having a SVGA resolution (800 pixels×606 pixels), and pixel size of 15 μm, corresponding to an active display area of 12 mm×9 mm, which, for preceding calculated focal distance, fₗₑₙₛ, of first lens assembly (L1a) 216, projects a retinal projection 290 having an area of 4 mm×3 mm across retina 162, as shown in FIG. 6a. Above described optical configuration corresponds to a field of view (FOV) 268 of 13.4°.

### Increasing (Expanding, Widening) the Field of View (FOV)

For implementing particular embodiments of the present invention which do not involve projecting a spatial pattern of at least about 5 μm onto fovea 164, then, for improving overall performance of the optical configuration of near eye module assembly (NEMa) 20 shown in FIG. 6a, there is need for increasing (expanding, widening) field of view (FOV) 268.

FIG. 6b is a schematic diagram illustrating an exemplary calculation of field of view (FOV) 268, without the 6/6 vision acuity design requirement shown in FIGS. 5a and 5b. As shown in FIG. 6b, for increasing (expanding, widening) field of view (FOV) 268, there is increasing the optical power of first lens assembly (L1a) 216 (in FIG. 6b, generally indicated as ‘effective’ lens, L_{1,2}, 264) by replacing the lens inside of first lens assembly (L1a) 216, or/and by inserting second lens assembly (L2a) 210 into incident optical path (IOP) 204. This procedure is combined with moving micro-display (μdisplay) 202 by means of micro-display distance regulator (μDDR) 232 to a new focal distance, i.e., focal distance, fₗₑₙₛ, 265. For example, using a lens with an effective optical power corresponding to a focal distance of 25 mm, there is increasing (expanding, widening) field of view (FOV)) 268 from 13.4° to 27°.

### Corneal Imaging

The optical configuration shown in FIG. 6a or 6b, used for projecting visual patterns onto retina 162, or/and for illuminating and imaging retina 162, is additionally utilized for imaging non-retinal eye structures, as shown in FIG. 6c, for example, for projection of special patterns onto, or/and imaging of, cornea 152. FIG. 6c is a schematic diagram illustrating an exemplary specific embodiment of an optical configuration suitable for corneal imaging, using near eye module assembly (NEMa) (20 in FIGS. 3a, 3b, and 3c; 20a or 20b, in FIG. 1), of the multi-functional optometric-ophthalmic system (10 illustrated in FIGS. 1 and 2).

As shown in FIG. 6c, ‘effective’ lens, L_{1,2}, 264, corresponding to an optical configuration including first lens assembly (L1a) 216, singly, or, optionally, in combination with second lens assembly (L2a) 210, is used together with refraction correction assembly (RCa) 218, and positioned relative to micro-display (μdisplay) 202 at a distance corresponding to twice the focal distance, fₗₑₙₛ, 265, of ‘effective’ lens, L_{1,2}, 264 (in FIG. 6c, this doubled focal distance is indicated by 293). Near eye module assembly (NEMa) 20 is positioned in front of eye 102 such that the distance between ‘effective’ lens, L_{1,2}, 264 and cornea 152 is also equivalent to the doubled focal distance 293.

### Multi-Axis Moving and Positioning Assembly (MMPa)

Head mountable unit 14, preferably, includes at least one multi-axis moving and positioning assembly 22, i.e., MMP assembly (MMPa) 22, where FIG. 1 shows head mountable unit 14 including four MMP assemblies, i.e., MMP assembly (MMPa) 22a, MMP assembly (MMPa) 22b, MMP assembly (MMPa) 26a, and MMP assembly (MAPa) 26b.

Multi-axis moving and positioning assembly (MMPa) 22 (i.e., 22a or 22b) is for moving and positioning of near eye module assembly (NEMa) 20 (i.e., 20a or 20b, respectively) relative to eye 102 of subject 12. Such moving and positioning is performed for up to six degrees of freedom, i.e., linear translation along the x-axis, the y-axis, or/and the z-axis; or/and rotation around (or relative to) the x-axis, the y-axis, or/and the z-axis. Multi-axis position assembly (MMPa) 22 (i.e., 22a or 22b) linearly moves and positions near eye module assembly (NEMa) 20 (i.e., 20a or 20b, respectively) in a range of, preferably, between about 0 centimeters (cm) and about 10 centimeters (cm) in each of the x-axis, the y-axis, or/and the z-axis, directions. Multi-axis position assembly (MMPa) 22 (i.e., 22a or 22b) rotationally or angularly moves and positions near eye module assembly (NEMa) 20 (i.e., 20a or 20b, respectively) in a range of, preferably, between about 0 degrees and about 180 degrees around (or relative to) each of the x-axis, the y-axis, or/and the z-axis, directions.

Multi-axis moving and positioning assembly (MMPa) 26 (i.e., 26a or 26b) is for moving and positioning of secondary fixation pattern assembly (SFPa) 24 (i.e., 24a or 24b, respectively) relative to eye 102 of subject 12. Such moving and positioning is performed for up to six degrees of freedom, i.e., linear translation along the x-axis, the y-axis, or/and the z-axis; or/and rotation around (or relative to) the x-axis, the y-axis, or/and the z-axis. Multi-axis position assembly (MMPa) 26 (i.e., 26a or 26b) linearly moves and positions secondary fixation pattern assembly (SFPa) 24 (i.e., 24a or 24b, respectively) in a range of, preferably, between about 0 centimeters (cm) and about 5 centimeters (cm) in each of the x-axis, the y-axis, or/and the z-axis, directions. Multi-axis position assembly (MMPa) 26 (i.e., 26a or 26b) rotationally or angularly moves and positions secondary fixation pattern assembly (SFPa) 24 (i.e., 24a or 24b, respectively) in a range of, preferably, between about 0 degrees and about 180 degrees around (or relative to) each of the x-axis, the y-axis, or/and the z-axis, directions.

### Secondary Fixation Pattern Assembly (SFPa)

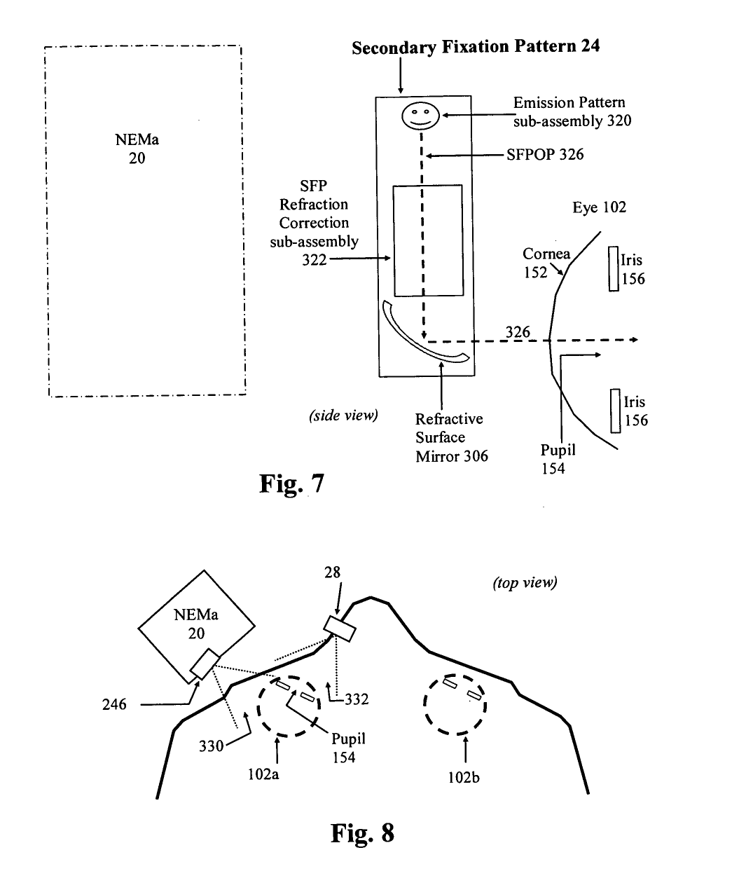

Head mountable unit 14, preferably, includes at least one secondary fixation pattern assembly 24, i.e., SFP assembly (SFPa) 24, where FIG. 1 shows head mountable unit 14 including two SFP assemblies, i.e., SFP assembly (SFPa) 24a and SFP assembly (SFPa) 24b. FIG. 7 is a schematic diagram illustrating a side view of an exemplary specific preferred embodiment of secondary fixation pattern assembly (SFPa) 24, and components thereof, as part of head mountable unit 14, of multi-functional optometric-ophthalmic system 10 illustrated in FIGS. 1 and 2. Illustrative description of the main functions (operations) of secondary fixation pattern assembly (SFPa) 24, and components thereof, with reference to FIG. 7, follows.

Secondary fixation pattern assembly (SFPa) 24 is for generating a fixation pattern for eye 102 of subject 12, for embodiments of the present invention wherein near eye module assembly (NEMa) 20 (i.e., 20a or/and 20b) is utilized for procedures or operations that do not involve generation of a primary fixation pattern for eye 102. Fixation of a specific target, for example, in the form of a pattern, is necessary for fixing the gaze of subject 12, in order to avoid eye movements, and accommodation, for the purpose of reducing complexities involved with different vision or eye examination procedures. An on-center positioned near eye module assembly near eye module assembly (NEMa) 20 (i.e., 20a or/and 20b) combines functions of retinal illumination and fixation pattern generation. However, when near eye module assembly (NEMa) 20 (i.e., 20a or/and 20b) is positioned off-center, due to imaging of off-centered angles of components of eye 102, then secondary fixation pattern assembly (SFPa) 24 is used for projecting a fixation pattern or target on retina 162, which is then fixed by fovea 164 of eye 102.

Secondary fixation pattern assembly (SFPa) 24 includes the main components of: (1) an emission pattern sub-assembly 320, (2) a secondary fixation pattern (SFP) refraction correction sub-assembly 322, and (3) a refractive surface mirror 324. The position of secondary fixation pattern assembly (SFPa) 24 relative to eye 102 and near eye module assembly (NEMa) 20 is shown in FIG. 7.

Emission pattern sub-assembly 320 is, preferably, a relatively small (‘tiny’) fixed pattern, for example, having size dimensions of about 2 millimeters (mm)×about 2 millimeters (mm), having any recognizable geometrical form or shape of some known object.

Secondary fixation pattern refraction correction sub-assembly 322 regulates or changes optical power of secondary fixation pattern assembly (SFPa) 24, to correspond to a refraction status of eye 102 which is measured by near eye module assembly (NEMa) 20. Alternatively stated, secondary fixation pattern refraction correction sub-assembly 322 is used for correcting or compensating optical power of secondary fixation pattern assembly (SFPa) 24, such that subject 12 can sharply see a fixation pattern, for example, emission pattern sub-assembly 320, which is perceived by subject 12 as being located far away from subject 12.

Refractive surface mirror 324 is used for providing a vertical optical path (in FIG. 7, indicated as SFPOP 326), of secondary fixation pattern assembly (SFPa) 24, in order to occupy least possible space between near eye module assembly (NEMa) 20 and eye 102. In general, refractive surface mirror 324 includes a reflective surface of essentially any geometrical shape, form, or configuration, which is suitable for functioning as a convex lens. Refractive surface mirror 324 includes, preferably, a reflective surface of a curved geometrical shape, form, or configuration, as shown in FIG. 7, for example, selected from the group consisting of a parabolic geometrical shape, form, or configuration; a hyperbolic geometrical shape, form, or configuration; and an elliptical geometrical shape, form, or configuration. For an embodiment of secondary fixation pattern assembly (SFPa) 24 which includes refractive surface mirror 324 including a reflective surface of a curved geometrical shape, form, or configuration, as shown in FIG. 7, then, via such curvature, optical power is increased, and there is precluding need for including additional lenses in secondary fixation pattern assembly (SFPa) 24. Alternatively, refractive surface mirror 324 includes a reflective surface of a non-curved (straight or flat) geometrical shape, form, or configuration, in combination with a lens (e.g., a convex type of lens).

### Fixed Imaging Assembly

Head mountable unit 14, preferably, includes at least one fixed imaging assembly 28, where FIG. 1 shows head mountable unit 14 including two fixed imaging assemblies, i.e., fixed imaging assembly 28a and fixed imaging assembly 28b. For an exemplary specific embodiment of multi-functional optometric-ophthalmic system 10 of the present invention, wherein head mountable unit 14 includes two fixed imaging assemblies, i.e., fixed imaging assembly 28a and fixed imaging assembly 28b, then, fixed imaging assembly 28a and fixed imaging assembly 28b are used for observing and imaging in and around the immediate regions of the left eye, and of the right eye, respectively.

Fixed imaging assembly 28 performs the same functions, and includes the same components as illustratively described hereinabove for mobile imaging assembly 246 (FIGS. 3a, 3b, 3c). Accordingly, as for mobile imaging assembly 246, fixed imaging assembly 28 is also for imaging anterior parts of eye 102, in particular, and for imaging facial anatomical features and characteristics in the immediate region of eye 102 of subject 12. Additionally, accordingly, fixed imaging assembly 28 includes the main components of: (1) a multi-spectral illumination source, (2) an imager, and (3) an electronically adjustable focus lens, each of which is illustratively described hereinabove with regard to mobile imaging assembly 246.

As the name of fixed imaging assembly 28 implies, fixed imaging assembly 28 is ‘fixed’ relative to eye 102, by way of being a fixed or stationary component mounted to head mounting assembly 18 of head mountable unit 14. This is in contrast to mobile imaging assembly 246, which is ‘mobile’ by being located and operative inside of mobile near eye module assembly (NEMa) 20. Such a basic difference between fixed imaging assembly 28 and mobile imaging assembly 246 has a significant implication regarding the different use and operation of these two components of multi-functional optometric-ophthalmic system 10, for testing, diagnosing, or treating, vision or eyes of a subject 12. Especially regarding imaging of anterior parts of eye 102, in particular, and for imaging facial anatomical features and characteristics in the immediate region of eye 102 of subject 12.

FIG. 8 is a schematic diagram illustrating a top view of an exemplary specific preferred embodiment particularly showing relative positions, and fields of view 330 and 332, of mobile imaging assembly 246 and fixed imaging assembly 28, in relation to facial anatomical features and characteristics in the immediate region of eye 102a of subject 12, for imaging thereof via multi-functional optometric-ophthalmic system 10 illustrated in FIGS. 1 and 2. As shown in FIG. 8, mobile imaging assembly 246 is located and operative inside of near eye module assembly (NEMa) 20, and has a field of view 330, whereas fixed imaging assembly 28 is located and operative outside of near eye module assembly (NEMa) 20, and has a field of view 332. Facial anatomical features and characteristics in the immediate region of eye 102a of subject 12 which are outside of field of view 330 of mobile imaging assembly 246, but are in field of view 332 of fixed imaging assembly 28, are only imagable by fixed imaging assembly 28. For example, as shown in FIG. 8, front portion of pupil 154 of eye 102a is outside of field of view 330 of mobile imaging assembly 246, but is in field of view 332 of fixed imaging assembly 28, and, therefore, is imagable by fixed imaging assembly 28.

### Analog Electronics Assembly (AEa)

Head mountable unit 14, preferably, includes analog electronics assembly 30, i.e., AE assembly (AEa) 30, as shown in FIG. 1. Analog electronics assembly (AEa) 30 is for interfacing and controlling integrated operation of head mountable unit 14 components which have analog electronic types of interfaces. Exemplary types of such components are motors without or with an encoder, variable focused liquid lenses, power supply circuit control devices, pinhole shutter and airpuff/ultrasound assembly 220, and electrode assemblies, such as sensoric electrodes assembly 44, and motoric electrodes assembly 46.

### Display Driver Assembly (DDa)

Head mountable unit 14, preferably, includes display driver assembly 32, i.e., DD assembly (DDa) 32, as shown in FIG. 1. Display driver assembly (DDa) 32 is for electronically driving micro-display (μdisplay) 202 of near eye module assembly (NEMa) 20.

Reference is again made to FIG. 1, for illustratively describing the structure and function (operation) of the various possible ‘optional’ components of head mountable unit 14 of multi-functional optometric-ophthalmic system 10. Head mountable unit 14, optionally, includes any number or combination of the following additional (optional) components: local controlling and processing assembly (LCPa) 34; digital signal processing assembly (DSPa) 36; audio means assembly (AMa) 38; power supply assembly (PSa) 40; position sensor assembly 42; sensoric electrodes assembly 44; and motoric electrodes assembly 46.

### Local Controlling and Processing Assembly (LCPa)

Head mountable unit 14, optionally, includes local controlling and processing assembly 34, i.e., LCP assembly (LCPa) 34, as shown in FIG. 1. Local controlling and processing assembly (LCPa) 34 is for ‘locally’ controlling and processing data and information relating to operation of the components (i.e., assemblies, sub-assemblies, etc.) of head mountable unit 14 of multi-functional optometric-ophthalmic system 10. Such controlling and processing is locally performed with respect to head mountable unit 14, and is distinguished from the central controlling and processing performed by central controlling and processing unit 16 of multi-functional optometric-ophthalmic system 10.

### Digital Signal Processing Assembly (DSPa)

Head mountable unit 14, optionally, includes digital signal processing assembly 32, i.e., DSP assembly (DSPa) 36, as shown in FIG. 1. Digital signal processing assembly (DSPa) 36 is for digital processing of video, image, or/and audio, types of data and information.

As stated, digital signal processing assembly (DSPa) 36 is optionally included in head mountable unit 14. In an alternative embodiment of multi-functional optometric-ophthalmic system 10, head mountable unit 14 is absent of digital signal processing assembly (DSPa) 36, and for alternatively performing the functions thereof, central controlling and processing unit 16 includes digital signal processing assembly (DSPa) 62. In another alternative embodiment of multi-functional optometric-ophthalmic system 10, head mountable unit 14 includes digital signal processing assembly (DSPa) 36, and for additionally performing the functions thereof, central controlling and processing unit 16 includes digital signal processing assembly (DSPa) 62.

### Audio Means Assembly (AMa)

Head mountable unit 14, optionally, includes audio means assembly 38, i.e., AM assembly (AMa) 38, as shown in FIG. 1. Audio means assembly (AMa) 38 is for transmitting (providing) instructions, or/and explanations, or/and essentially any other type or kind of audio information, to subject 12, for example, via digital to analog (D/A) converters, amplifiers, and speakers. Audio means assembly (AMa) 38 is also for receiving verbal responses from subject 12, for example, via microphones, amplifiers, and analog to digital (A/D) converters. Following such reception, audio means assembly (AMa) 38 sends digitized verbal responses to digital signal processing assembly 32, i.e., DSP assembly (DSPa) 32, which performs automatic speech recognition.

### Power Supply Assembly (PSa)

Head mountable unit 14, optionally, includes power supply assembly 40, i.e., PS assembly (PSa) 40, as shown in FIG. 1. Power supply assembly (PSa) 40 is for supplying power to head mountable unit 14.

As stated, power supply assembly (PSa) 40 is optionally included in head mountable unit 14. In an alternative embodiment of multi-functional optometric-ophthalmic system 10, head mountable unit 14 is absent of power supply assembly (PSa) 40, and for alternatively performing the functions thereof, central controlling and processing unit 16 includes power supply assembly (PSa) 60. In another alternative embodiment of multi-functional optometric-ophthalmic system 10, head mountable unit 14 includes power supply assembly (PSa) 40, and for additionally performing the functions thereof, central controlling and processing unit 16 includes power supply assembly (PSa) 60.

Power supply assembly (PSa) 40 is based on standard 110 volt/220 volt, alternating current (AC), types of electrical power supplies. Alternatively, or additionally, power supply assembly (PSa) 40 is based on disposable battery, direct current (DC), types of electrical power supplies or/and rechargeable battery, direct current (DC), types of electrical power supplies.

### Position Sensor Assembly

Head mountable unit 14, optionally, includes position sensor assembly 42, as shown in FIG. 1. Position sensor assembly 42 is for detecting, indicating, and monitoring, changes in global (coordinate) positions of head mountable unit 14, which are associated with same such changes in global (coordinate) positions of the head of subject 12. This association of changes in global (coordinate) positions of head mountable unit 14 with the head of subject 12 is the direct result of head mounting assembly 18 firmly and securely mounting head mountable unit 14 upon the head of subject 12, in accordance with the preferred embodiments of multi-functional optometric-ophthalmic system 10.

Specific examples of operation of position sensor assembly 42 are for detecting, indicating, and monitoring, changes in global (coordinate) positions of head mountable unit 14 due to, and associated with, changes in global (coordinate) positions of the head during examination or treatment of head gaze coordination, or during head movements associated with implementing the present invention according to a virtual reality application.

### Sensoric Electrodes Assembly

Head mountable unit 14, optionally, includes and sensoric electrodes assembly 44, as shown in FIG. 1. Sensoric electrodes assembly 44 is for sensing a visual evoked potential (VEP) in the visual cortex area of the brain of subject 12. Such visual evoked potential (VEP) is associated with operation of head mountable unit 14, while performing examinations or tests of vision of subject 12, such as automatic vision acuity examinations or tests, or automatic vision fields examinations or tests. In an exemplary specific embodiment of head mountable unit 14, sensoric electrodes assembly 44 is mounted upon band strips that are secured to the scalp region associated with the visual cortex area.

### Motoric Electrodes Assembly

Head mountable unit 14, optionally, includes and motoric electrodes assembly 46, as shown in FIG. 1. Motoric electrodes assembly 46 is for sensing electrical potentials which arise due to activity of the frontal cortex area of the brain of subject 12, for the purpose of activating intra- and extra-ocular muscles of eye 102. In an exemplary specific embodiment of head mountable unit 14, motoric electrodes assembly 46 is mounted upon band strips that are secured to the scalp region associated with the frontal cortex area.

Reference is again made to FIG. 1, for illustratively describing the structure and function (operation) of central controlling and processing unit 16, and, components and functionalities thereof, as part of multi-functional optometric-ophthalmic system 10.

### Central Controlling and Processing Unit

In multi-functional optometric-ophthalmic system 10, central controlling and processing unit 16 is for overall controlling and processing of functions, activities, and operations, of head mountable unit 14.

Central controlling and processing unit 16, preferably, includes any number or combination of the following components: control assembly 50, operator input assembly 52, display assembly 54, subject input assembly 56, communication interface assembly (CIa) 58, and power supply assembly (PSa) 60, as schematically shown in FIG. 1.

### Control Assembly

In central controlling and processing unit 16, control assembly 50 is for overall controlling of multi-functional optometric-ophthalmic system 10, for testing, diagnosing, or treating, vision or eyes of subject 12, by operator 15. Such overall controlling includes running of the operating system (OS), software programs, software routines, software sub-routines, software symbolic languages, software code, software instructions or protocols, software algorithms, or/and a combination thereof.

Such overall controlling also includes running of hardware used for implementing the present invention, such as electrical, electronic or/and electromechanical system units, sub-units, devices, assemblies, sub-assemblies, mechanisms, structures, components, and elements, and, peripheral equipment, utilities, accessories, and materials, which may include one or more computer chips, integrated circuits, electronic circuits, electronic sub-circuits, hard-wired electrical circuits, or/and combinations thereof, involving digital or/and analog operations.

### Operator Input Assembly

In central controlling and processing unit 16, operator input assembly 52 is for inputting or entering, into control assembly 50, data and information about or associated with subject 12, by operator 15. Operator input assembly 52 is also for inputting or entering, into control assembly 50, data and information associated with controlling of multi-functional optometric-ophthalmic system 10, and the various components and functions thereof, by operator 15. Operator input assembly 52 is, for example, an integrated set of a computer keyboard and mouse.

### Display Assembly

In central controlling and processing unit 16, display assembly 54 is for displaying previously described data and information which has been input or entered into control assembly 50, by operator 15. Display assembly 54 is also for displaying data and information which has been input or entered into control assembly 50, and is directed to subject 12, for the purpose of training subject 12 regarding the various vision or eye examinations or tests, or treatments, implemented by using multi-functional optometric-ophthalmic system 10, and the methodologies thereof.

### Subject Input Assembly

In central controlling and processing unit 16, subject input assembly 56 is for inputting or entering, into control assembly 50, commands or/and responses by subject 12, in response to interacting with the various vision or eye examinations or tests, or treatments, implemented by using multi-functional optometric-ophthalmic system 10, and the methodologies thereof. Such interactive commands or/and responses input or entered by subject 12 is associated with training or actual vision or eye examinations or tests, or treatments provided by the present invention.

Subject input assembly 56 is, for example, a joystick type device or mechanism, particularly designed, constructed, and operative, for equivalent use by right and left hands, or for simultaneous use by both hands, of subject 12, and for specific needs or requirements of multi-functional optometric-ophthalmic system 10, and the methodologies thereof. In an exemplary specific embodiment of the present invention, such a joystick type device or mechanism, is particularly designed, constructed, and operative, for right hand or/and left hand inputting or entering, into control assembly 50, commands or/and responses, by subject 12, which are correspondingly associated with the respective right eye or/and left eye, of subject 12.

Communication Interface assembly

In central controlling and processing unit 16, communication interface assembly 58, i.e., CI assembly (CIa) 58, is for interfacing control assembly 50 of multi-functional optometric-ophthalmic system 10 with external equipment, devices, utilities, accessories, or/and networks. Exemplary types of interfacing are based on universal serial bus (USB), ethernet, wireless fidelity (WiFi), cellular (e.g., global system for mobile communications (GSM)), types of communication technologies.

### Power Supply Assembly

In central controlling and processing unit 16, power supply assembly 60, i.e., PS assembly (PSa) 60, is for supplying power to central controlling and processing unit 16. In an alternative embodiment of multi-functional optometric-ophthalmic system 10, head mountable unit 14 is absent of power supply assembly (PSa) 40, and for alternatively performing the functions thereof, power supply assembly (PSa) 60 of central controlling and processing unit 16 supplies power to both control and processing unit 16 and to head mountable unit 14. In another alternative embodiment of multi-functional optometric-ophthalmic system 10, head mountable unit 14 includes power supply assembly (PSa) 40, for supplying power to head mountable unit 14, and central controlling and processing unit 16 includes power supply assembly (PSa) 60 for supplying power to and central controlling and processing unit 16.

Power supply assembly (PSa) 60 is based on standard 110 volt/220 volt, alternating current (AC), types of electrical power supplies. Alternatively, or additionally, power supply assembly (PSa) 60 is based on disposable battery, direct current (DC), types of electrical power supplies or/and rechargeable battery, direct current (DC), types of electrical power supplies.

Central controlling and processing unit 16, optionally, includes any number or combination of the following additional (optional) components: a digital signal processing assembly 62, herein, also referred to as DSP assembly (DSPa) 62; and a pneumatic pressure generator assembly 64.

### Digital Signal Processing Assembly

In central controlling and processing unit 16, (optional) digital signal processing assembly 62, i.e., DSP assembly (DSPa) 62, is for digital processing of video, image, or/and audio, types of data and information.

As stated, digital signal processing assembly (DSPa) 62 is optionally included in central controlling and processing unit 16. In an alternative embodiment of multi-functional optometric-ophthalmic system 10, central controlling and processing unit 16 is absent of digital signal processing assembly (DSPa) 62, and for alternatively performing the functions thereof, head mountable unit 14 optionally includes digital signal processing assembly (DSPa) 36. In another alternative embodiment of multi-functional optometric-ophthalmic system 10, central controlling and processing unit 16 includes digital signal processing assembly (DSPa) 62, and for additionally performing the functions thereof, head mountable unit 14 includes digital signal processing assembly (DSPa) 36.

### Pneumatic Pressure Generator Assembly

In central controlling and processing unit 16, (optional) pneumatic pressure generator assembly 64 is for generating pneumatic pressure which is transferred, via high pressure air transfer line 65, to air pressure distributor 306 (FIG. 4b) of pinhole shutter and airpuff/ultrasound assembly 220, for distributing an air pressure wave (i.e., an airpuff), via near eye module assembly (NEMa) 20, to cornea 152 of eye 102 of subject 12. Transference of the pneumatic pressure is effected, and controlled, by a release valve included in pneumatic pressure generator assembly 64 of central controlling and processing unit 16, or/and by a release valve included in air pressure distributor 306 of pinhole shutter and airpuff/ultrasound assembly 220.

### Procedures and Methodologies for Operating the Multi-Functional Optometric-Ophthalmic System

The corresponding method for testing, diagnosing, or treating, vision or eyes of a subject, of the present invention, includes the following main steps or procedures, and, components and functionalities thereof: (a) mounting head mountable unit 14, upon the head of subject 102, wherein head mountable unit 14 includes: (i) head mounting assembly 18, for mounting assemblies of multi-functional optometric-ophthalmic system 10 upon the head of subject 102; and (ii) at least one near eye module assembly (NEMa) 20 (i.e., near eye module assembly (NEMa) 20a or/and near eye module assembly (NEMa) 20b, mounted upon head mounting assembly 18, for generating optical processes or effects which act or take place upon, and are affected by, at least one eye of subject 12, and for receiving results of the optical processes or effects from at least one eye 102, as part of the testing, diagnosing, or treating of the vision or eyes of subject 12, wherein each near eye module assembly includes the various components as illustratively described hereinabove; and (b) controlling and processing of functions, activities, and operations, of components of head mountable unit 14, by central controlling and processing unit 16, operatively connected to head mountable unit 14.

For implementing the method of the present invention, including performing each of the hereinbelow described procedures for operating multi-functional optometric-ophthalmic system 10, there is using near eye module assembly (NEMa) 20 (i.e., 20a or/and 20b), which is moved and positioned by multi-axis moving and positioning assembly (MMPa) 22 (i.e., 22a or/and 22b, respectively). Controlling any of the hereinbelow described procedures is performed by local controlling and processing assembly (LCPa) 34 of head mountable unit 14, or/and by control assembly 50 of central controlling and processing unit 16, while processing of data or/and information is performed by digital signal processing assembly (DSPa) 36 of head mountable unit 14, or/and by digital signal processing assembly (DSPa) 62 of central controlling and processing unit 16.

### Mapping of Facial Anatomy

After mounting head mountable unit 14 of multi-functional optometric-ophthalmic system 10 on the head of subject 12, multi-axis moving and positioning assembly (MMPa) 22 moves and positions near eye module assembly (NEMa) 20 in front of a distant facial position. Next, the facial geometry is captured by means of mobile imaging assembly 246 of each near eye module assembly (NEMa) 20 and three dimensional (3-D) facial data and information is extracted and recorded. This data and information is further used by multi-functional optometric-ophthalmic system 10 for optimally moving and positioning near eye module assembly (NEMa) 20 and secondary fixation pattern assembly (SFPa) 24, according to facial characteristics of subject 12, and according to requirements of each specific procedure.

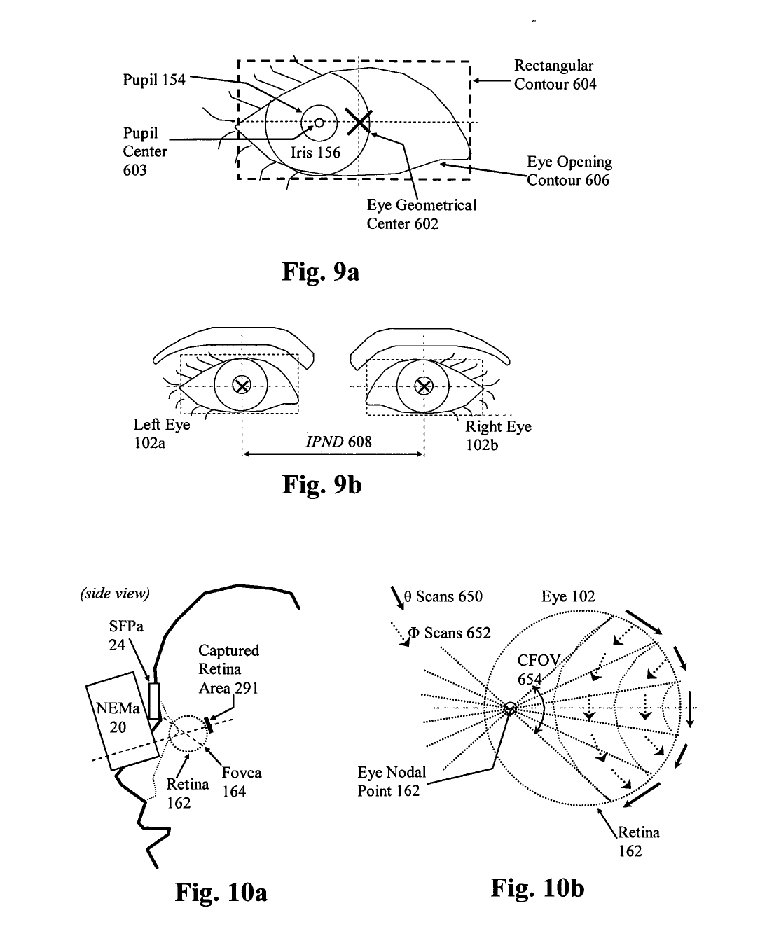

Near Eye Module Assembly Position Initialization and External Measurements Once head mountable unit 14 is mounted on the head of subject 12, and its facial anatomy has been mapped, the initial position of near eye module assembly (NEMa) 20 is adjusted such that micro-display (μdisplay) 202 is centered at the geometrical center of eye 102, as shown in FIG. 9a. The control of location of near eye module assembly (NEMa) 20 (i.e., 20a or/and 20b) relative to the eye position is performed following processed image data and information received from mobile imaging assembly 246.

Since there is no guarantee that head mounting assembly 18 is symmetrically mounted on the head of subject 12, therefore, each near eye module assembly (NEMa) 20 is individually adjusted according to the same distance and position relative to eye 102. Initial position of each near eye module assembly (NEMa) 20 is done respectively to geometrical center of eye 602 (FIG. 9a) that lies on the same incident optical path (IOP) 204 with the center of the micro-display (μdisplay) 202. This procedure provides geometrical parameters, such as the eye opening contour 606 and ‘Inter Pupilary Normal Distance’ (IPND) 608 (FIG. 9b).

### Refraction Correction Adjustment

Refraction correction adjustment is performed according to either a manual mode, or according to an automatic mode. For each mode, optical power of lenses inside refraction correction assembly (RCa) 218 is updated, or refraction power is updated by changing position of micro-display (μdisplay) 202, by means of micro-display distance regulator (μDDR) 232. The procedure is performed according to either a monocular mode, or a binocular mode.

In a manual mode, refractive power is adjusted by subject 12, or/and by operator 15, according to feedback sent by subject 12 by means of subject input assembly 56. In an automatic mode, refractive power is adjusted using retinal imaging received through reflection optical path (ROP) 222 and algorithm that finds best correlation between the test pattern, transmitted along incident optical path (IOP) 204 and the image reflected from the retina 162 of eye 102 of subject 12 and transmitted back through reflection optical path (ROP) 222 to imager 228, see details in ‘Retinal Illumination Visual Stimuli Focusing and Position Securing’ procedure. Once best correlation is achieved, the algorithm slowly increases the refractive power of the refraction correction assembly (RCa) 218. The increase is done until a correlation exists, which means that there is a decrease in accommodation of intra-ocular lens 158 of eye 102 of subject 12. Once lens 158 reaches its flatness limit, the correlation is decreased, and at this point the algorithms stops. This enables revealing of a fine refraction condition adjustment for distant objects.

### Visual Stimulation

The procedure of visual stimulation performed using near eye module assembly (NEMa) 20, which is moved and positioned by multi-axis moving and positioning assembly (MMPa) 22. Alternatively, this operation is performed by means of secondary fixation pattern assembly (SFPa) 24 which position is controlled by MMPa 26.

This procedure follows previously described ‘Refraction Correction Adjustment’ procedure. Once refraction for subject 12 was adjusted, the vision stimulation is used to take attention of subject 12 to fixate and follow fixation object. This fixation object is generated either by micro-display (μdisplay) 202 of near eye module assembly (NEMa) 20, or by secondary fixation pattern assembly (SFPa) 24. Usually the fixation object is at normal intensity to the human vision which is about 60 cd/m².

There are two possibilities to change position of the visual stimuli. The first option is more suitable for near eye module assembly (NEMa) 20, where the position of the fixation object is changed on the micro-display (μdisplay) 202. The second option is changing position of near eye module assembly (NEMa) 20 by means of MMP assembly (MMPa) 22 or, alternatively, change position of the secondary fixation pattern assembly (SFPa) 24 by means of MMP assembly (MMPa) 26.

### Eye Tracking

For performing of the eye or pupil tracking procedure, mobile imaging assembly 246 of near eye module assembly (NEMa) 20 or/and fixed imaging assembly 28 are used.

Once the eye 102 of the subject 12 is stimulated by fixation pattern it is used by procedures to ensure that subject's eye 102 follows the fixation pattern. This is performed by means of mobile imaging assembly 246 of near eye module assembly (NEMa) 20 or/and fixed imaging assembly 28 by capturing the video of the eye, processing by means digital signal processing assembly (DSPa) 32 or 62 and detection the center of the pupil 603 (FIG. 9a). For each location of visual stimuli the eye tracking algorithm calculates expected location of the center of pupil 603. The eye tracking procedure reports difference between locations of expected and actual centers of pupil 603.

### Retinal Illumination Visual Stimuli Focusing and Position Securing

This procedure is performed in combination of near eye module assembly (NEMa) 20, which is moved and positioned by multi-axis moving and positioning assembly (MMPa) 22 and secondary fixation pattern assembly (SFPa) 24 which position is controlled by MMPa 26.

This procedure utilizes both functionalities of the micro-display (μdisplay) 202 that are: (1) generation of normal intensity patterns, pictures, or/and videos and (2) short interval pulses (e.g., on the order of milliseconds (ms)) of high intensity pattern or illumination. The short interval high intensity pulses are short enough not to be perceived by human nervous system and from other side intense enough such that retina reflections could be imaged by means of imager 228. The total energy of those pulses is not hazardous to the human eye.

The procedures requiring short, intense pulses generated by micro-display (μdisplay) 202 could be classified as following: (i) illumination of retina 162 of eye 102 for retinal imaging presented in ‘Retinal Photography and Scanning for Ultra-Wide Field of View’ procedure; (ii) ‘Visual Stimulations’ used in ‘automatic visual acuity test’ and ‘visual fields examination’.

For every abovementioned procedure focus and location of high intensity pattern or illumination generated by micro-display (μdisplay) 202 should be secured on retina 162 of eye 102 of subject 12, such that influence of intra-ocular lens 158 accommodation and eye 102 motion will be tolerable. This requirement is achieved by performing procedure, described in the current section, through short time period (less than 20 msec) for which effect of intra-ocular lens 158 accommodation and eye 102 motion is not significant.

The protocol for the ‘Retinal Illumination Visual Stimuli Focusing and Position Securing’ procedure is as following:
(i) Generating normal intensity visual stimulus using ‘Visual Stimulation’ procedure by means of near eye module assembly (NEMa) 20 or secondary fixation pattern assembly (SFPa) 24.

For the following steps near eye module assembly (NEMa) 20 only is used. The following steps are performed in as short time interval for which effect of intra-ocular lens 158 accommodation and eye 102 motion is not significant.
(ii) Adjusting refraction correction assembly (RCa) 218 such that the stimulus is focused on the retina 162 of eye 102 of subject 12.
(iii) Generating short, intense, pulse for uniform illumination of the retina 162 of eye 102 of subject 12. Capture the retinal image by means of imager 228, analyze the retinal image and figure out the retinal region the picture is covering.
(iv) Adjusting near eye module assembly (NEMa) 20 position to new position covering necessary region of the retina 162 of eye 102 of subject 12 by repeating step (iii) until necessary position is achieved.
(v) Generating visual stimulus of intensity, duration and pattern corresponding to particular requirement of vision or eye test or examination. This stimulus is generated by micro-display (μdisplay) 202 that selectively activate pixels respectively to stimulus area and exact location on the retina 162 of eye 102 of subject 12.

### Retinal Photography and Scanning for Ultra-Wide Field of View

This procedure is performed in combination of near eye module assembly (NEMa) 20, which is moved and positioned by multi-axis moving and positioning assembly (MMPa) 22 and secondary fixation pattern assembly (SFPa) 24 which position is controlled by MMPa 26.

Retinal photography utilizes procedure described in ‘Retinal Illumination Visual Stimuli Focusing and Position Securing’ procedure. In the example of calculation of filed of view (FOV) 268, (FIG. 6b) of near eye module assembly (NEMa) 20 used for imaging of retina 162 of eye 102 of subject 12 filed of view (FOV) 268 is about 27°. This section describes procedure of utilization of head mountable unit 14 resources for covering major area of the retina 162 of eye 102 of subject 12.

The resource used for covering most of retina 162 of eye 102 of subject 12 area are near eye module assembly (NEMa) 20, which is precisely moved and positioned by multi-axis moving and positioning assembly (MMPa) 22 and secondary fixation pattern assembly (SFPa) 24 which position is controlled precisely by MMPa 26 (FIG. 10a). The coordinates of imaged area of retina 162 of eye 102 of subject 12 are precisely extracted using combination of ‘Retinal Illumination Visual Stimuli Focusing and Position Securing’ procedure and ‘Eye Tracking’ procedure. Therefore, consequent areas of retina 162 of eye 102 of subject 12 are stitched together. The stitching creates combined field of view (CFOV) 654 solid angle using two axes scans: θ scans 650 and Φ scans 652 as illustrated on FIG. 10b.

### Monocular Distance Perception Regulation

The ‘Monocular Distance Perception’ (MDP) of virtual objects perceived by subject 12 is regulated by means of changing optical power of the Refraction Correction assembly (RCa) 218 or by regulating micro-display (μdisplay) 202 distance from first lens assembly (L1a) 216.

First, ‘Refraction Correction Adjustment’ procedure is performed. The intra-ocular lens 158 of eye 102 of subject 12 is at released condition respectively to the condition that eye 102 of subject 12 fixates emulated distant object following the procedure of ‘Refraction Correction Adjustment’. The change in distance perception, in monocular mode, is going along with activation of accommodation of intra-ocular lens 158 of eye 102 of subject 12. The accommodation is activated by addition of negative refraction power by means of Refraction Correction assembly (RCa) 218 or by regulating micro-display (μdisplay) 202 distance from first lens assembly (L1a) 216.

### Eye Movement Stimulation Binocular Fixation and Distance Perception and Position Regulation

This procedure is performed by near eye module assemblies (NEMa) 20a and 20b, which are moved and positioned by multi-axis moving and positioning assembly (MMPa) 22. Alternatively, this procedure is performed by secondary fixation pattern assemblies (SFPa) 24b and 20b which positions are controlled by MMPa 26 and 26b.

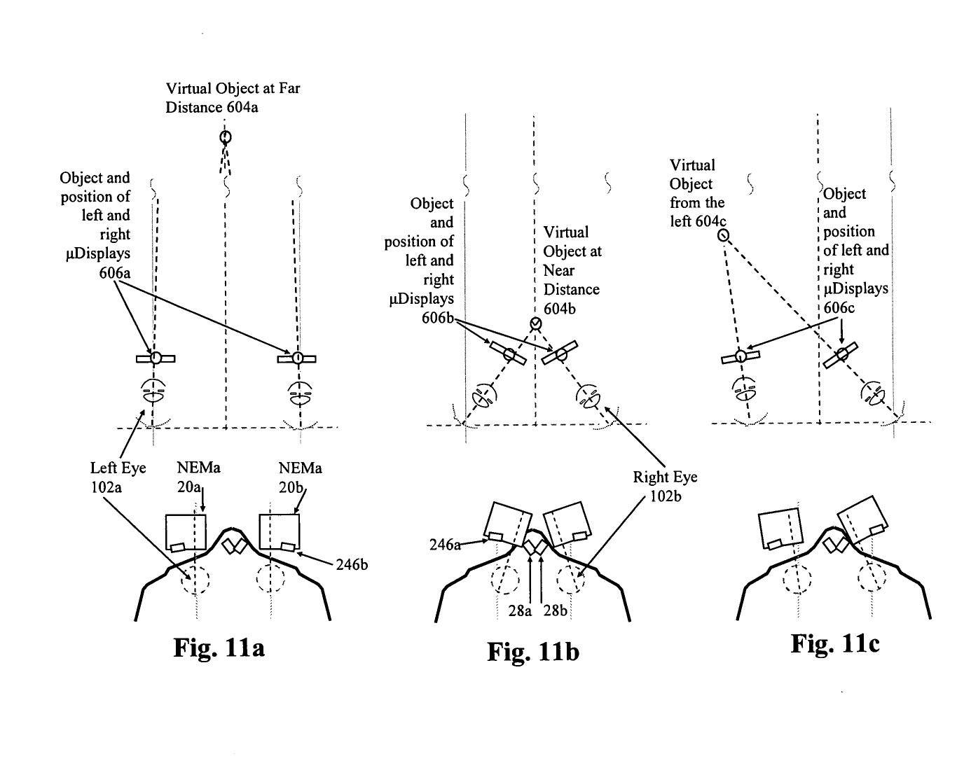

First ‘Near Eye Module Assembly Position Initialization and External Measurements’ procedure is performed. Near eye module assemblies (NEMa) 20a and 20b are positioned straight in front of eyes 102a and 102b of subject 12 (FIG. 11a). Next, ‘Refraction Correction Adjustment’ procedure is performed for left and right eyes 102 of subject 12. Following those two procedures subject 12 is expected to fuse similar objects 606a placed on optical axis for every eye as shown on FIG. 11a to single object illustrated on FIG. 11a as virtual object at far distance 604a. This fuse of the similar objects presented two both eyes is known as binocular fixation.

The emulation of object location distance in binocular mode is performed using combination of ‘Monocular Distance Perception Regulation’ procedure and ‘Visual Stimulation’ procedure such that near eye module assemblies (NEMa) 20a and 20b are moved and appropriately positioned. Virtual object at near distance 604b is emulated by respective visual stimuli generation represented by 606b as illustrated on FIG. 11b. Virtual object from the left 604c is emulated by respective visual stimuli generation represented by 606c as illustrated on FIG. 11c.

### Prisms Emulation

The procedure of prisms emulation is performed using near eye module assembly (NEMa) 20, which is moved and positioned by multi-axis moving and positioning assembly (MMPa) 22. Alternatively, this operation is performed by means of secondary fixation pattern assembly (SFPa) 24 which position is controlled by MMPa 26.

Prisms are often used by optometrists to check phorias/tropias of the subject 102. The deviations from normal conditions are measured in prismatic diopters. Near eye module assembly (NEMa) 20 introduces prisms by either prismatic type correction of refraction correction assembly (RCa) 218 or by emulation prism emulation.

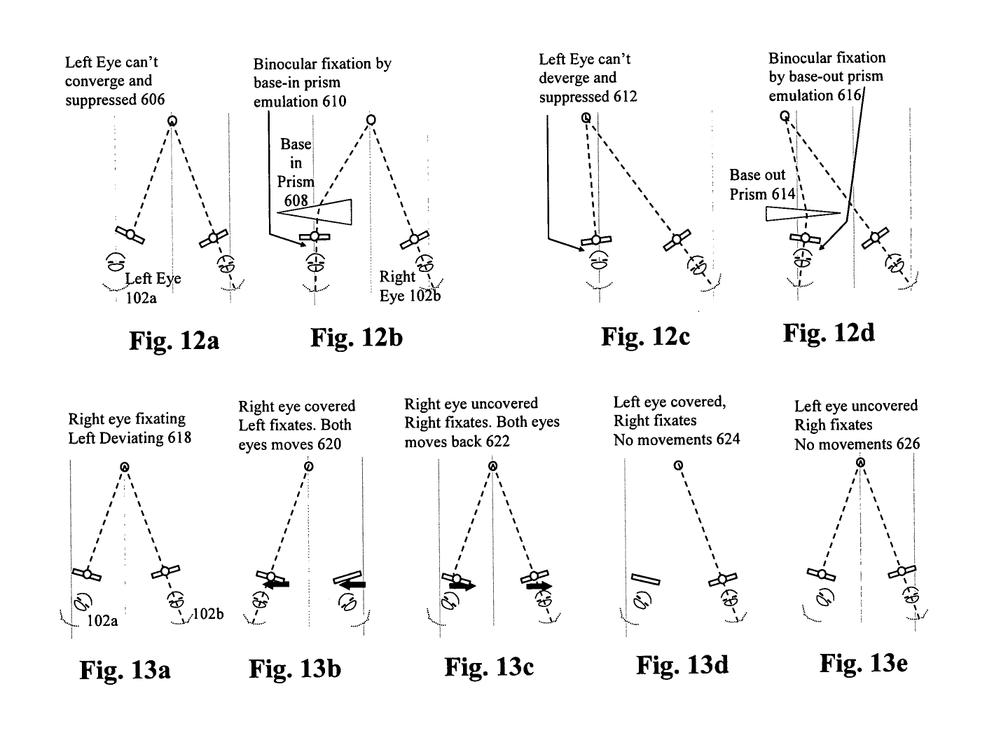

The prism emulation illustrated on FIG. 12b and FIG. 12d. FIG. 12a illustrates an inability to converge and resulted suppression 606 of left eye 102a of subject 12. Binocular fixation is recovered for subject 12 by emulation of base in prism 608 by shift of visual stimulus 610 as illustrated on FIG. 12b. An example of inability to diverge and resulted suppression 612 of left eye 102a of subject 12 is illustrated on FIG. 12c. Binocular fixation is recovered for subject 12 by emulation of base out prism 614 by shift of visual stimulus 616 as illustrated on FIG. 12d.

### Cover Test

The procedure of cover test is performed using near eye module assembly (NEMa) 20, which is moved and positioned by multi-axis moving and positioning assembly (MMPa) 22. Alternatively, this operation is performed by means of secondary fixation pattern assembly (SFPa) 24 which position is controlled by MMPa 26. The ‘Eye Tracking’ procedure is used along the ‘Cover Test’ procedure.

The Phoria condition of strabismus is tested by a “Cover Test” that is actually occlusion of one of the eyes. Depending on the phoria eyes condition the eyes moves from fixed position when one of them occluded and moving again when cover is removed. In prior art the cover test performed manually. In this section we present objective and automatic way for performing the cover test by means of a multi-functional optometric-ophthalmic system 10.

The cover test procedure is exemplified by sequence illustrated on FIG. 13a through FIG. 13e. First binocular object is emulated using ‘Eye Movement Stimulation Binocular Fixation and Distance Perception and Position Regulation’ procedure. Situation of fixating right eye and deviating left eye 618 is shown on FIG. 13a. The emulation of right eye 102b occlusion is illustrated on FIG. 13b. The emulation of occlusion is done by turning off of micro-display (μdisplay) 202b. As shown on FIG. 12b, both eyes moves to left 620. The eyes movement is measured using ‘Eye Tracking’ procedure. Following ‘removing of occlusion’ from right eye 102b, by turning on micro-display (μdisplay) 202b, right eye 102b fixates again and both eyes are moved to the right 622 (FIG. 13c). Next, left eye 102a is occluded, right eye 102b continues fixating and no eyes movement is detected 624 (FIG. 13d). Following ‘removing of occlusion’ from left eye 102a, by turning on micro-display (μdisplay) 202a, no change in eyes position is detected 626 (FIG. 13e).

Progressive Projection of Patterns onto the Cornea

This procedure is performed in combination of near eye module assembly (NEMa) 20, which is moved and positioned by multi-axis moving and positioning assembly (MMPa) 22 and secondary fixation pattern assembly (SFPa) 24 which position is controlled by MMPa 26.

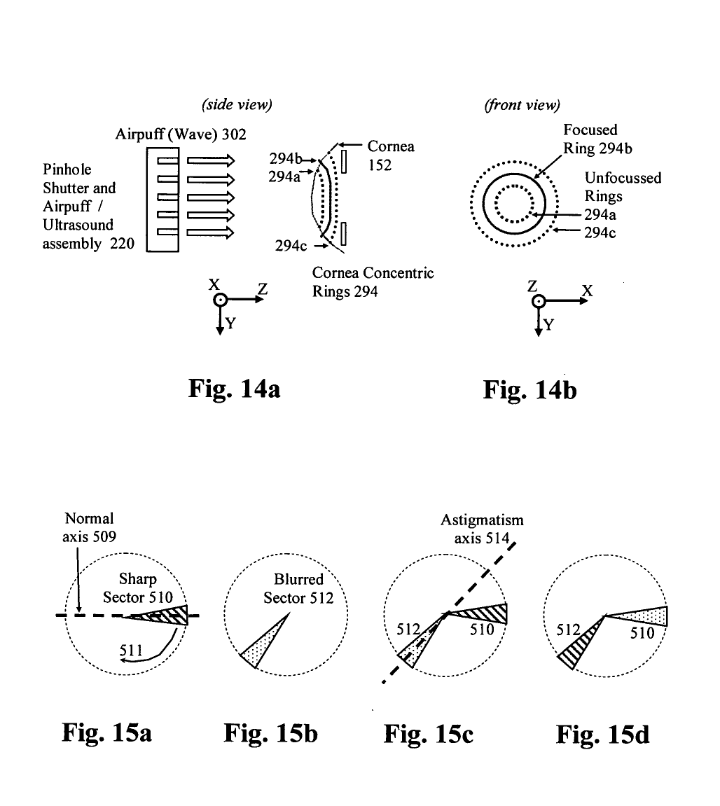

The ‘Progressive Projection of Patterns onto the Cornea’ procedure is used for example for corneal topography, for corneal or iris imaging, intra-ocular pressure measurement and for cornea thickness mapping. The optical setup configuration for ‘Progressive Projection of Patterns onto the Cornea’ was presented on FIG. 6c. Using MMP assembly (MMPa) 22 the focus plane on cornea surface could be progressively regulated. FIG. 14 and FIG. 14b illustrates the surface that in focus, exemplified by focused concentric ring 294b on the cornea 152 of eye 102 of subject 12. Two additional surfaces are of focus 294a and 294c.

For corneal topography deformation of concentric rings is imaged and three dimensional (3-D) information is extracted. Secondary fixation pattern assembly (SFPa) 24 is used for reduce movements of eye 102 of subject 12. The concentric rings and deformation imaging are done progressively respectively with the increase of the diameter of concentric rings 294a, 294b and 294c.

The intra-ocular pressure measurement is done using airpuff wave 302 generated by pinhole shutter and airpuff/ultrasound assembly 220 (FIG. 14a). Concentric rings 294a, 294b and 294c are projected simultaneously while only one could be in focus. Due to deformation of cornea 152 of eye 102 of subject 12 by airpuff wave 302 the focus passes from one ring to another. The transition of focus is corresponds to deformation of cornea 152 of eye 102 of subject 12 and intra-ocular pressure is calculated since it corresponds to the deformation of cornea 152 of eye 102 of subject 12 too.

For cornea thickness mapping position of near eye module assembly (NEMa) 20 that corresponds to each concentric ring in focus is measured twice during progression. The distance between first and second condition of focus for specific concentric ring, along Z-axis, indicates corneal thickness in the corresponding region of cornea 152 of eye 102 of subject 12.

The same, progressive focusing is done for fluorescence and spectral imaging in case that depth of focus of one-shot case is not satisfactory. Finally, the information received from progressive procedure is stitched together.

### Astigmatism Diagnosis Procedure

This procedure is performed by near eye module assembly (NEMa) 20, which is moved and positioned by multi-axis moving and positioning assembly (MMPa). This procedure is useful for the structure of refraction correction assembly (RCa) 218 that not includes cylindrical correction optics. The procedure could be performed manually, using input response from subject 12 through subject input assembly 56 or automatically using automatic mode of ‘Refraction Correction Adjustment’ procedure.

First, correct refraction condition for subject 12 is adjusted using ‘Refraction Correction Adjustment’ procedure. Next, test pattern in form of 1/18 of circle, for example, is generated in the center of micro-display (μdisplay) 202 of near eye module assembly (NEMa) 20. This form referred as a sector as shown on sequence of figures: FIG. 15a through FIG. 15d. The sharp sectors as shown on FIG. 15a as sharp sector 510 are relates to normal axis 509. This sharp sector 510 is rotated until turns to be blurred 512 (FIG. 15b). The position of blurred sector 512 is corresponds to astigmatism axis 514 and sharp sector 510 is presented again (FIG. 15c). Refraction power is adjusted by means of emulation or by means of refraction correction assembly (RCa) 218 until sharp sector 510 turns to be blurred and blurred sector 512 turns to be sharp (FIG. 15d). This refraction power corresponds to cylindrical power of astigmatism.

Immediately following are illustrative descriptions of several exemplary specific preferred embodiments of implementing the present invention, for testing, diagnosing, or treating, vision or eyes of a subject. Throughout the following illustrative description, it is to be clearly understood that each of the various different exemplary specific preferred embodiments of examinations correspond to different exemplary specific preferred embodiments of implementing the ‘same’ generalized system, and the ‘same’ corresponding generalized method thereof, according to the present invention, and do not correspond to different, unrelated or/and independent systems or methods.

### Exemplary Vision or Eye Tests (Examinations)

Each of the hereinbelow described vision or eye examinations is performed by local controlling and processing assembly (LCPa) 34, or by control assembly 50, while image and information processing is performed by digital signal processing assembly (DSPa) 36 or 62.

The structure of multi-functional optometric-ophthalmic system 10 and its procedures used as a platform for implementation of almost any vision examination procedure existing in optometry, ophthalmology and vision neurology practice. The examples of vision examinations and treatment most commonly used in practice are provided. For convenience, the vision examinations are classified in three categories:

(i) Automatic—examinations not requiring cooperation of the examinee.

(ii) Objective—examinations where results depend on cooperation of examinee. This cooperation almost always is fixation of gaze on fixation target.

(iii) Subjective—examinations that completely dependent on feedback from examinee.

### Automatic Tests or Examinations

### Fundus Photography

An example of scanning sequence is shown in FIG. 10b. We defined there θ scans and Φ scans such that for every θ scan a sequence of Φ scans is performed. A combined field of view (CFOV) 654 of about 130° could be covered in about 1+2×3+2×5=17 scans. The procedure takes about half minute. Each retinal imaging capturing, that includes focusing and position securing procedure, takes about half second and the rest of time is necessary to move near eye module assembly (NEMa) 20 to required positions to perform θ and Φ scans.

### Angiography

The angiography is performed in the same way as regular fundus photography with appropriate set of fluorescence filters (excitation and emission) selected inside near eye module assembly (NEMa) 20. Depending on fluorescine material the appropriate excitation filter is selected in micro-display filters assembly (μDFa) 208 and emission filter is selected in imager filters assembly (IFA) 226.

### Oximetry

The oximetry is performed in the close way to the regular fundus photography combined with a spectral imaging. The spectral imaging is achieved either by filtering white light of the micro-display (μdisplay) 202 by means of selection of appropriate filter from micro-display filters assembly (μDFa) 208. Or by filtering by means of appropriate filter of imager filters assembly (IFA) 226 the white light reflected from retina 162 of eye 102 of subject 12.

### Electro-Physiology Tests

For this test the procedure ‘Visual Stimulation’ procedure is used in combination with ‘Retinal Illumination Visual Stimuli Focusing and Position Securing’ procedure and with the sensoric electrode 44 of near eye module assembly (NEMa) 20.

The electro-physiology tests utilize neurological feedback of the vision system. They allow performing prompt and precise assessment of central and peripheral vision. The tests are based on stimulation of photoreceptors and measuring “Visual Evoked Potentials” (VEP) in a visual cortex are by means of sensoric electrodes assembly 44. The tests use ‘Retinal Illumination Visual Stimuli Focusing and Position Securing’ procedure such that precise mapping if VEP responses is done.

### Automatic Vision Acuity and Color Test

Central vision is performed by photoreceptors of the macula region 166 (FIG. 3a). The highest vision acuity achieved by usage of central vision. In addition, color vision could be achieved by usage of central vision too. The ability of EVE to project visual stimulus of spot of 5×1.6 μm, using optical configuration on FIG. 6c in combination with sub-pixel activation, allows stimulating of almost single cone.

The VEP measurement from single stimulation takes about ½ sec. For scanning entire macula the following example of optimization could be applied. First macula 166 is stimulated and scanned under low resolution and then suspicious regions are scanned under high resolution.

The VEP response on the visual stimulations is performed either using white light, normally used for visual acuity test, or using specific color preselected by means of μDFa 208 of near eye module assembly (NEMa) 20.

### Automatic Visual Fields Testing

The setup used on FIG. 10a is used. The automatic Visual fields testing performed by using secondary fixation pattern assembly (SFPa) 24 (stimulating gaze to track the pattern) and high intensity point flashes, generated by near eye module assembly (NEMa) 20, stimulating peripheral vision.

### Visual Axis Opacities Detection

Opacities in the visual axes detectable by “Red Reflex” test. Retina 162 of eye 102 of subject 12 is the colorless, tissue paper-thin layer of cells. Underneath the transparent retina is another layer of the eye that provides the nourishment to the retina. This thin blood filled layer is called the choroid, and is reddish-orange in color.

Bright, white light uniform illumination, is generated by means of micro-display (μdisplay) 202, and is projected onto 162 of eye 102 of subject 12. The light reflected off of the choroid produces a red-orange (or sometimes orange-yellow) image on the imager 228 for healthy eyes (and this is called the “Red Reflex”).

If anything interferes with the transmission of light through the front of the eye—to the rear and back again (i.e. intra-ocular lens 158 or vitreous body 160—FIG. 3a), the reflex is affected, producing either a white (light bouncing off something white inside the eye) or black (no light getting in to bounce back) reflex rather than red-orange. First the eyes assessed separately, than are viewed together. Any asymmetry in color, brightness, or size is an indication for referral, because asymmetry may indicate an amblyogenic condition.

### Photophobia Diagnostics

This test is performed using micro-display (μdisplay) 202 of near eye module assembly (NEMa) 20 and fixed imaging assembly 28 and/or mobile imaging assembly 246.

Photophobia, or light sensitivity, is an intolerance of light. The main symptom of photophobia is discomfort in bright light and a need to squint or close eyes to escape it. The light level is gradually increased by micro-display (μdisplay) 202 and eyes response is tracked by fixed imaging assembly 28 and/or mobile imaging assembly 246.

### Objective Tests or Examinations

The objective vision examination requires cooperation of subject 12, while feedback or subject 12 response is registered by multi functional optometric-ophthalmic system 10 automatically. Subject 12, in most cases, has to follow fixation pattern only.

### Refraction Status

For this test, ‘Refraction Correction Adjustment’ procedure or/and ‘Astigmatism Diagnosis’ procedure is/are used.

### Accommodation Amplitude

For this test, ‘Monocular Distance Perception’ procedure is used.

### Eye Movements

For this test, ‘Visual Stimulation’ and ‘Pupil Tracking’ procedures are used.

Eye 102 of subject 12 movements could be divided on dynamic and static. For static movements the ability to bring eye to certain position is tested. This ability is depends on one or more of six extra-ocular muscles. For the dynamic movement velocity of movements of subject's 12 eyes 102 are examined. The involuntary movements are examined as well.

### Extra-Ocular Muscles Test

There six extra-ocular muscles responsible to move eye 102 and also provide slight rotations. The test patterns are generated such that being followed by eyes to cardinal positions that are straight ahead (primary position), straight up, down, left and right, and up/left, up/right, down/left and down/right. The eyes are evaluated in their abilities to look in all 9 cardinal positions of gaze, when examined individually and jointly.

### Oculomotor Skills and Nystagmus

Oculomotor Skills is the ability to quickly and accurately move the eyes. They necessary to move eyes so we can direct and maintain a steady visual attention on an object (fixation), move eyes smoothly from point to point as in reading (saccades), and track a moving object (pursuits) efficiently.

Testing Ocular Motility enables to differentiate between: comitant (remains constant with gaze direction) versus incomitant (varies in size with the direction of gaze) disorders. The tests could be binocular or monocular. For monocular test one eye is inactive (black background is projected), however its movement is still tracked by ‘pupil tracking’ procedure. The test patterns are generated and moved in a saccadic and pursuit way. Subject's 12 pupils 154 are tracked and their movements are analyzed.

The cyclotorision movements of eye 102 are detectable too. A number of reference points fixed on iris 156 so if eye have been rotated it's distinguishable according to position of reference points on iris 156.

For saccadic test the text could be generated word by word or letter by letter where subject 12 requested to read and pronounce the text. The speech of subject 12 is captured by audio means assembly 38 and processed for correctness of text that he has read along with the saccadic eye movements. For pursuits different moving patterns could be generated while the patient can regulate the speed of movements, by means of subject input assembly 56 such, that he still fixates single object (no diplopia). During the saccades and/or the pursuits the latent nystagmus could be revealed. Nystagmus is assessed by performing ‘Pupil Tracking’ procedure at sampling frequency of around two hundred hertz.

### Eyes Teaming

For these tests, ‘Eye Movement Stimulation Binocular Fixation and Distance Perception and Position Regulation’ procedures are used.

Cover Test for Phoria/Tropia Assessment Sensory and motor fusion mechanisms ensure a correct alignment of eyes to allow binocular vision. If the sensory fusion is prevented, e.g. by occlusion of one eye, motor fusion will be frustrated and a deviation of the visual axes will occur in many patients. If the motor fusion reflex eliminates the deviation when the obstacle to sensory fusion is removed, the deviation is latent, and is called a phoria. The cover test for phoria/tropia assessment is performed using the ‘Cover Test’ procedure.

### Near Point of Convergence Assessment

The ‘Near Point of Convergence’ (NPC) is assessed using procedure ‘Eye Movement Stimulation Binocular Fixation and Distance Perception and Position Regulation’ combined with procedure for Eye Tracking. First, distant binocular vision is emulated and next to that the emulated distance is decreased. The eyes 102a and 102b of subject 12 are converging and are tracked by mobile imaging assembly 246 and/or fixed imaging assemblies 28. When 102a and 102b of subject 12 are stops converging and can't follow the emulated, approaching, object the ‘Near Point of Convergence’ (NPC) is figured out.

### Pupillary Reflexes and Hippus

The assessment of the pupil provides a relatively quick and easy, objective assessment of visual function that requires little patient co-operation and should therefore be incorporated into every eye examination. Pupillary misosis is a function of accommodation, vergence and illumination. The illumination level is controlled by means the micro-display (μdisplay) 202, while for accommodation control we use ‘Monocular Distance Perception’ (MDP) procedure and for vergence using procedure ‘Eye Movement Stimulation Binocular Fixation and Distance Perception and Position Regulation’. The diameters and responses of pupils 154a and 154b of left eye 102a and right eye 102b, respectively, are measured by means of fixed imaging assembly 28 or/and mobile imaging assembly 246.

### Pupillary Light Reflex

The main function of the iris 156 is to control the amount of light entering the eye 102 and reaching the retina 162. It also protects the visual pigments from bleaching. Therefore reaction on flash light will be pupil miosis known as ‘Pupil Light Reflex’ (PLR). The receptors of the PLR are the retinal rods and cones and it is unlikely that specific ‘pupillary’ receptors exist.

### Pupillary Vergence Reflex

For the Pupillary Vergence Reflex a binocular fixation object at specific distance is emulated. This binocular fixation object has low intensity on black background. After that, the intensity of the background for left eye 120b, is increased. This results in constriction of the left eye 120b, while the right eye 120a follows the left 120b (a consensual response). The same procedure performed for the right eye 102b.

### Pupillary Accommodation Reflex

To check pupillary response as function of accommodation, monocular target is used. The distance perception is changed and pupil's diameter is tracked correspondingly. After monocular assessment of first eye the same procedure is repeated for the second. The procedure is performed for constant illumination. In binocular case, pupillary responses as function of vergence are measured under constant illumination condition while binocular distance perception is changed.

### Pupillary Hippus Reflex

For this test, ‘Progressive Projection of Patterns onto the Cornea’ and ‘Eye Tracking’ procedures are used.

Physiological pupillary hippus (oscillation) measurement gives a useful measure of visual function. The oscillation frequency is lower in optic nerve lesions and following the use of barbiturates. The oscillation elicited by illumination of pupil margin. It is done by using ‘Progressive Projection of Patterns onto the Cornea’ in order to project bright pattern on the perimeter of the pupil. The changes in pupil 154 geometry are captured using ‘Eye Tracking’ procedure. Near eye module assembly (NEMa) 20 is positioned of axis such that secondary fixation pattern assembly (SFPa) 24 is used to generate fixation pattern for the eye. The tracked changes in pupil 154 geometry are actually oscillations (pupil constriction and redilataion) which period is calculated as average for, for example, 100 oscillations.

### Confrontation Visual Fields Test

The ‘Confrontation Visual Fields Test’ (CVFT) is an objective, precise and fast procedure for vision fields' assessment. It is performed by means of secondary fixation pattern assembly (SFPa) 24 and near eye module assembly (NEMa) 20 and it is monocular procedure. Two sources of vision stimuli exist in the CVFT setup. First, subject 102 fixates object generated by secondary fixation pattern assembly (SFPa) 24. He is requested to switch his gaze on the second object, generated by near eye module assembly (NEMa) 20 once it is appears. The near eye module assembly (NEMa) 20 is initially positioned at one of cardinal gaze angle position. The primary fixation pattern, generated by near eye module assembly (NEMa) 20, is moved slowly toward central part of eye 102 until subject 102 switches fixation from secondary fixation pattern assembly (SFPa) 24 to primary fixation pattern. When such “fixation switch” occurs, the maximal visual filed for particular direction is figured out.

### Objective Vision Acuity Test (Gratings on Gray Card)

The objective vision acuity could be assessed using “Grating on Gray Card” (GGC). The gray background is generated by micro-display (μdisplay) 202. Gratings of low spatial frequency, corresponding to 20/200 vision acuity, is generated first and randomly moves through the micro-display (μdisplay) 202. The grating spatial frequency increases until subject 12 tracks the grating. The procedure continues until the subject 12 can fixate the fixation grating. In such way the vision acuity is evaluated objectively.

### Subjective Tests or Examinations

For the subjective tests or examinations, the feedback received from subject 12 through either ‘subject input assembly’ 56 or through audio means assembly 38. Display assembly 54 is used to train subject 12 to use subject input assembly 56 before actual test. Multi-functional optometric-ophthalmic system 10 allows to subject 12 selection of the answer from a reduced number of options. The selection is performed using subject input assembly 56 or through audio means assembly 38.

### Subjective Vision Acuity, Refraction and Contrast Sensitivity

The subjective vision acuity and subjective refraction tests are combined. The procedure is performed first in monocular way and then for both eyes simultaneously. First, target such as, for example, Snellen Chart is presented. Refraction correction assembly (RCa) 218 is set for extreme value, +15D for instance. The subject 12 changes the dioptic power by means of subject input assembly 56 until best vision acuity is achieved. Next, subject 12 selects the last row that he still can see sharply. In such way refraction status and vision acuity are evaluated simultaneously.

Additional visual acuity tests could be done. Those are any standard test known in optometry and ophthalmology such as LH symbols (LEA symbols) and Allen cards, the tumbling E test, and the HOTV test.

Next, pinhole test could be repeated using pinhole shutter and airpuff/ultrasound assembly 220. The Pinhole shutter is positioned close to the eye by means of frontal distance regulator (FDR) 244. If results of vision acuity are better for the pinhole test, there is some unresolved refraction problem, otherwise, if the vision acuity reduced from 6/6, an amblyopia or other retina related conditions could be suspected.

In all conditions where visual acuity is reduced, contrast sensitivity is reduced as well. In some conditions that reduce vision acuity, contrast sensitivity is reduced more than expected based upon the visual acuity alone. Therefore after vision acuity is measured, contrast sensitivity test is performed for the last vision target. Let's take tumbling E test for example. The vision acuity test was done using black background and white letter (or vice versa). Now the contrast between background and letter is reduced until the subject 12 losses ability to indicate the “E” letter direction.

### Binocular Fixation Convergence and Suppression and Diplopia

For this test or examination, virtual circle is generated at infinity using ‘Eye Movement Stimulation Binocular Fixation and Distance Perception and Position Regulation’. In a case that subject 12 perceives two circles or deformed circle—diplopia is suspected. Then circles are changed to square to one eye 102a and triangle to the second eye 102b. Using the subject input assembly 56 subject 12 requested to bring rectangle into the square. The shift distance of the both objects corresponds to ‘Amount of Disparity’ between the eyes 102a and 102b of subject 12. This shift is equivalent to addition of prism as described in ‘Prisms Emulation’ procedure.

The procedure could be combined with ‘Cover Test’ in order to evaluate suppression in the case that no diplopia is suspected. Further, the procedure could be combined with ‘Visual Stimuli Focusing and Position Securing’ procedure, in order to find exactly degree of eccentric fixation by evaluating the shift of fixating point from the fovea. The last option allows Micro-Strabismus detection.

### Stereopsys

Standard Stereopsis test are implemented on the system. The 3D pictures like ‘stereo-fly’ are presented and the subject 12 should indicate, through either ‘subject input assembly’ 56 what he sees by selection from objects palette. Subject 12 selects the most prominent object.

### Subjective Color Test

Micro-display (μdisplay) 202 generates uniform, large test patterns white patterns. The color generated by selection of appropriate filter in micro-display filters assembly (μDFa) 208. In addition a palette of colors is presented on the micro-display (μdisplay) 202 area covered by red-green-blue filter assembly (RGBFa) 206. The subject 12 selects best matching color from virtual colors palette.

### Examples of Vision or Eye Therapy—Treating Vision or Eyes of the Subject

The ‘Natural Vision Therapy’ (NVT) cares the eyes and brain simultaneously. Multi-functional optometric-ophthalmic system 10 is a highly effective for NVT of visual disorders categories such as: (1) lazy eye (amblyopia); (2) crossed eyes (strabismus); (3) vergence and accommodation problems; (4) anomalous retinal correspondence (ARC), suppressions and double vision (diplopia). It also useful for some reading and learning disabilities where it is specifically directed toward resolving visual problems which interfere with reading, learning and educational instruction. In addition, the eye related neurological disorders could be treated by corresponding nerve stimulation.

### Vergence, Accommodation, and Acculomotion Management

The effective strabismus and amblyopia management requires elimination refraction errors, vergence, accommodation and aculomotion disorders first. After elimination of the refraction errors the treatment for the missing visual skills development could be started.

For this therapy ‘Monocular Distance Perception’ procedure is used in combination with ‘Eye Tracking’ procedure.

The accommodation exercises are performed by changing monocular distance perception of virtual objects stimulating accommodation of the subject's 12 eye 102.

For vergrence ‘Eye Movement Stimulation Binocular Fixation and Distance Perception and Position Regulation’ is used. The patient gets pursuits and saccades exercise the same as during vergence diagnostics oriented to strengthen the weak muscles and improve eye teaming. For the occulomotion skills development, the patient gets exercises to move eyes through cardinal points.

### Amblyopia Management

For those therapy ‘Eye Movement Stimulation Binocular Fixation and Distance Perception and Position Regulation’ procedures are used in combination with ‘Eye Tracking’ procedure.

Amblyopia is a degradation of sensitivity of foveal light receptors (mostly cones) or brain related pathways. Multi-functional optometric-ophthalmic system 10 resources are used to stimulate the fovea 164. The treatments include monocular and binocular procedures. The fovea region detected by means of retinal imaging or according pupil central visual axis. Correct refraction conditions should be adjusted first in order to focus object on the macula 166.

### Non-Strabismic Amblyopia

For non strabismic amblyopia the management is quite simple. The main goal is enforcement of the amblyopic eye to work. It's trivial for monocular procedure, while for binocular the video divided on central part streamed to amblyopic eye and peripheral part for dominating eye. This could be movie, game or exercise combined with accommodation and vergence management procedures. Color vision disorders could be stimulated by generating images of badly perceptible color in the central region of the video.

### Strabismic Amblyopia

In a case of amblyopia along with strabismus the brain discarding information from stabismic eye in order to suppress double vision (diplopia) by suppression. During monocular vision the amblyopic eye tracks objects by means of eccentric fixation. The main goal is to redirect the fixation point back to the fovea 164. It could be done using pleoptics technique. An afterimage is generated by means of flashing the normal eye such that only foveal region is not shaded. This could be done suing strong flashes generated by micro-display (μdisplay) 202 of about 20 msec durations. Events like objects, games or movies are generated in frame having dimensions of non-shaded region on the second screen that corresponds to the problematic eye. A placement of the events on the screen is central in order to be associated with the fovea of a normal eye. In order to see them clearly the patient will have to use the fovea of the problematic eye, otherwise the events will be shaded. This process stimulates the fovea 164 to take back the fixation and regenerate sensitivity.

Other possibility is to ask patient to fixate on some object while flashing another bright object into fovea 164. If patient moves eye 102 the flashing object on the micro-display (μdisplay) 202 changes position correspondingly. This process is stimulates the brain to use fovea 164 for fixation.

### Strabismus Management

For those therapy ‘Eye Movement Stimulation Binocular Fixation and Distance Perception and Position Regulation’ procedures are used in combination with ‘Eye Tracking’ procedure.

The origin of strabismus could be due to eccentric fixation or eye muscles imbalance or both of them. Therefore corresponding factor are treated. In case that there is only eccentric fixation the starbismic ambliopya is managed. If there are an eye muscles disorders occulomotion skills are being developed to return ability for central fixation. High deviations of tropia are treated by surgery that decease the amplitude of deviation. The low deviations are treated by pursuits and saccades. The pursuits and saccades are monocular since for strabismic non-amblyopic eye confusions and diplopia will occur in case of binocular vision. The object movements are generated in a manner corresponding to training the problematic muscles of the eye.

The present invention, as illustratively described and exemplified hereinabove, has several beneficial and advantageous aspects, characteristics, and features, which are based on or/and a consequence of, the above illustratively described main aspects of novelty and inventiveness.

Based upon the above indicated aspects of novelty and inventiveness, and, beneficial and advantageous aspects, characteristics, or features, the present invention successfully overcomes several significant limitations, and widens the scope, of presently known techniques of testing, diagnosing, or treating, vision or eyes of a subject. Moreover, the present invention is readily industrially applicable.

It is appreciated that certain aspects and characteristics of the invention, which are, for clarity, described in the context of separate embodiments, may also be provided in combination in a single embodiment. Conversely, various aspects and characteristics of the invention, which are, for brevity, described in the context of a single embodiment, may also be provided separately or in any suitable sub-combination.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the scope of the appended claims.

### REFERENCES

1. Schweigerling, J., Field Guide to Visual and Ophthalmic Optics, Publisher: SPIE—The International Society for Optical Engineering, Washington, USA (2004).
2. U.S. Pat. Appl. Pub. No. US 2005/0002113 A1, to Berge, entitled: “Drop Centering Device”.
3. U.S. Pat. No. 6,369,954, to Berge et al., entitled: “Lens With Variable Focus”.

1-107. (canceled)

108. An eye-testing system, comprising:
a head mountable unit;
a near eye module assembly, movably mounted on said head mountable unit and having a microdisplay, an imager, and optics for directing light from said microdisplay to an eye of a subject and from said eye to said imager;
a mobile imaging assembly for providing image data of said eye; and
a central controlling and processing unit, configured for processing said image data, determining a geometrical center of said eye and positioning said near eye module assembly such that said geometrical center and said microdisplay are on an optical path defined by said optics.

109. The method of claim 108, wherein said central controlling and processing unit is configured for processing retinal image data generated by said imager and controlling said optics so as to secure focus of light generated by said microdisplay on said retina.

110. The system of claim 108, wherein said positioning said near eye module is performed automatically.

111. The system of claim 108, further comprising a secondary fixation pattern assembly for generating emission patterns selected such as to fix a gaze of said eye to a predetermined virtual location, said secondary fixation pattern assembly being movable relative to said near eye module assembly.

112. The system of claim 111, wherein said secondary fixation pattern assembly comprises a refraction correction sub-assembly configured for regulating optical power of said secondary fixation pattern assembly to correspond to a refraction status of said eye.

113. The system of claim 108, comprising a left near eye module assembly for a left eye and a right near eye module assembly for a right eye, wherein said central controlling and processing unit is configured for performing an optometric or ophthalmic eye-test in a binocular manner.

114. The system of claim 113, comprising:
a left secondary fixation pattern assembly for generating emission patterns selected such as to fix a gaze of said left eye to a first predetermined virtual location; and
a right secondary fixation pattern assembly for generating emission patterns selected such as to fix a gaze of said right eye to a second predetermined virtual location;
wherein each of said left and said right secondary fixation pattern assemblies is movable relative to a respective near eye module assembly.

115. The system of claim 113, wherein said central controlling and processing unit is configured for performing a strabismus test while controlling optics of each of said near eye module assemblies so as to secure focus of light generated by a respective microdisplay on a retina of a respective eye.

116. The system of claim 115, wherein said central controlling and processing unit is configured for providing said left and said right eyes with a visual stimulus at plurality of virtual locations, wherein said strabismus test is performed for each virtual location of said plurality of virtual locations.

117. The system of claim 108, wherein said optics comprises a refraction correction assembly, and wherein an optical power associated with said refraction correction assembly is controllable by said central controlling and processing unit to adjust a state of refraction of said eye while receiving light from said microdisplay.

118. The system of claim 108, wherein said near eye module assembly is configurable for corneal imaging.

119. The system of claim 118, wherein said central controlling and processing unit is configured for processing corneal images so as to map thickness and topography of said cornea.

120. The system of claim 119, wherein said central controlling and processing unit is configured for progressively regulating a focus of light generated by said microdisplay on said cornea, so as to obtain three-dimensional information of said cornea thereby to map said thickness and said topography of said cornea.

121. The system of claim 119, wherein said near eye module assembly includes a pinhole shutter and an air pressure wave assembly for applying pressure onto said cornea.

122. The system of claim 121, wherein said central controlling and processing unit is configured for calculating intra-ocular pressure by correlating said applied pressure with said three-dimensional information of said cornea.

123. The system of claim 117, further comprising a rotatable mirror for optically gating a reality window thereby exposing or blocking said eye to or from a real environment outside said near eye module assembly, wherein said central controlling and processing unit is configured for controlling said optical power of said refraction correction assembly to adjust said state of refraction of said eye while receiving light from said real environment.

124. The system of claim 108, further comprising a sensoric electrodes assembly for sensing a visual evoked potential in the visual cortex area of a brain of said subject.

125. The system of claim 108, wherein said near eye module assembly comprises a first filter assembly positioned for filtering light generated by said microdisplay, and a second filter assembly positioned for filtering light entering said imager.

126. The system of claim 108, wherein said central controlling and processing unit is configured for signaling said near eye module assembly to move and image a retina of said eye from a plurality of different orientations with respect to said eye to provide a plurality of retinal images, and stitching said plurality of retinal images to provide a combined image of said retina.

127. An eye-testing system, comprising:
a head mountable unit;
a left and right near eye module assemblies, each being movably mounted on said head mountable unit and having a microdisplay, an imager, and optics for directing light from said microdisplay to a respective eye of a subject and from said eye to said imager; and
a central controlling and processing unit, configured for processing image data generated by imagers of said left and right near eye module assemblies and performing a refractive condition test and a vision acuity test for both eyes simultaneously.

128. The system of claim 127, wherein said central controlling and processing unit is configured for performing at least one additional eye-test selected from the group consisting of strabismus test, convergence amplitude test and pupillary reflexes.

129. The system of claim 127, wherein said central controlling and processing unit is configured for performing eyes training for therapy of amblyopia and/or strabismus and/or accommodation abilities.

130. The system of claim 127, wherein said optics comprises a refraction correction assembly, and wherein an optical power associated with said refraction correction assembly is controllable by said central controlling and processing unit to adjust a state of refraction of a respective eye while receiving light from a respective microdisplay.

131. The system of claim 130, wherein each near eye module assembly comprises a rotatable mirror for optically gating a reality window thereby exposing or blocking a respective eye to or from a real environment outside said near eye module assembly, wherein said central controlling and processing unit is configured for controlling said optical power to adjust a state of refraction of a respective eye while receiving light from said real environment.

132. The system of claim 127, further comprising a secondary fixation pattern assembly for generating emission patterns selected such as to fix a gaze of said eye to a predetermined virtual location said secondary fixation pattern assembly being movable relative to said near eye module assembly.

133. The system of claim 127, further comprising a left and a right mobile imaging assemblies for respectively providing image data of said left eye and said right eye, wherein said central controlling and processing unit is configured for processing said image data, determining a geometrical center of each eye and positioning a respective near eye module assembly such that said geometrical center and said microdisplay are on an optical path defined by said optics.

134. An eye-testing system, comprising:
a head mountable unit;
a near eye module assembly, movably mounted on said head mountable unit and having a microdisplay, an imager, and optics for directing light from said microdisplay to an eye of a subject and from said eye to said imager, said microdisplay being capable of generating light rays having any wavelength from about 400 nanometers to about 1000 nanometers;
a central controlling and processing unit, configured for performing an optometric or ophthalmic eye-test by processing image data generated by said imager.

135. An eye-test method, comprising:
mounting on a head of a subject a head mountable eye-testing system which comprises a head mountable unit and a near eye module assembly (NEMa) movably mounted on said head mountable unit;
operating said NEMa so as to provide an eye of said subject with a visual stimulus while imaging a retina of said eye and securing focus of said stimulus on said retina; and
performing at least three optometric or ophthalmic eye-tests.

136. The method of claim 135, wherein said at least three optometric or ophthalmic eye-tests are selected from the group consisting of refractive condition, vision acuity, accommodation amplitude, pupillary reflexes, pupillary hippus, color test, extra-ocular muscles, cornea surface, tearing of eye, visual axis opacity, corneal topography, corneal thickness, oculomotor skills, nystagmus, fundus photography and visual field assessment.

137. The method of claim 135, wherein said head mountable eye-testing system comprises a left NEMa and a right NEMa for providing virtual stimuli to a left eye and a right eye, respectively, and wherein said at least three optometric or ophthalmic eye-tests are performed in a binocular manner.

138. The method of claim 135, further comprising:
providing image data of said eye,
processing said image data so as to determine a geometrical center of said eye, and
positioning said near eye module such that said geometrical center and said microdisplay are on an optical path defined by said optics.

139. The method of claim 135, further comprising opening a reality window in said near eye module assembly to expose said eye to a real environment outside said near eye module assembly.

140. The method of claim 135, wherein said visual stimulus is provided through a refraction correction assembly, and the method further comprising changing an optical power associated with said refraction correction assembly so as to emulate change in monocular distance of said visual stimulus while securing focus of said stimulus on a retina of said eye.

141. The method of claim 137, further comprising performing binocular fluorescence retina imaging.

142. The method of claim 137, further comprising changing a position of said visual stimuli while securing focus of each of said stimuli on a retina of a respective eye, and performing eye-track procedure to determine diameter, position and/or motion of a pupil of said eye, thereby determining convergence of said eyes in response to said change of position of said stimuli, thereby emulating change in binocular distance and position.

143. The method of claim 137, wherein each of said visual stimuli is provided through a refraction correction assembly, and the method further comprising:
changing an optical power associated with each refraction correction assembly so as to emulate change in monocular distance of a respective visual stimulus while securing focus of said stimulus on a retina of a respective eye;
changing a position of said visual stimuli while securing focus of each of said stimuli on a retina of a respective eye while performing eye-track procedure to determine diameter, position and/or motion of a pupil of said eye, thereby determining convergence of said eyes in response to said change of position of said stimuli; and
repeating said changing of said optical power and said changing of said position until said convergence is stopped, thereby determining a near point of convergence for said subject.

144. The method of claim 142, further comprising changing illumination level of said virtual stimulus while changing said position of said visual stimuli.

145. The method of claim 135, wherein said visual field assessment comprises:
operating a secondary fixation pattern assembly for generating emission patterns selected such as to fix a gaze of said eye to a predetermined virtual location;
operating said NEMa to provide said eye with a peripheral vision stimulus; and
performing an eye-track procedure to determine response of said eye to said peripheral vision stimulus, thereby assessing said visual field.

146. The method of claim 145, further comprising receiving signals pertaining to a visual evoked potential in the visual cortex area of a brain of said subject, and correlating said visual evoked potential to said peripheral vision stimulus.
